(19) United States
(12) Patent Application Publication
(10) Pub. No.: US 2010/0072686 A1
(43) Pub. Date: Mar. 25, 2010

(54) CONDOM HOLDER AND A METHOD OF PUTTING THE CONDOM ON

(76) Inventor: Alexander Leonidovich FELITSYN, Saint Petersburg (RU)

Correspondence Address: Dr. Maria Nilova;PATENTICA LLC, Office 303, Sadovaya Street 51, Saint Petersburg, 190068, RU

(21) Appl. No.: 12/626,698

(22) Filed: Nov. 27, 2009

Related U.S. Application Data
(63) Continuation-in-part of application No. PCT/RU06/00719, filed on Jun. 11, 2008.

(30) Foreign Application Priority Data
Jun. 14, 2007 (RU) 2007123536
Jun. 11, 2008 (RU) PCT/RU2008/000381

(51) Int. Cl. B25B 9/00; A61F 6/04

(52) U.S. Cl. 269/2; 128/844

(57) ABSTRACT

A condom holder is suggested along with a method of putting on the condom with said holder.

A condom holder at least partially defining a space in which the flatly arranged condom can be placed, that comprises fasteners used for fixating the condom edge; a connection between said fasteners that includes two opposite parts, each of which includes at least one of said fasteners; a third part placed between said parts that provides the moveability of said opposite parts at least in opposite directions; and a device used to displace said opposite parts using the mechanical force applied by the user; said third part is formed in such manner that said moveability of opposite parts in opposite directions includes the possibility of rotation thereof.

The method and the device of the invention allow the user to unpack and put on the condom in a quick and comfortable way, only slightly distracting the user from sexual intercourse

### CROSS-REFERENCE TO RELATED APPLICATIONS

This application claims the benefits from International Application PCT/RU2008/000381 filed on Jun. 11, 2008 and priority application RU 2007123536 filed on Jun. 14, 2007. The contents of these applications are hereby incorporated by reference and in their entirety.

### FIELD OF THE INVENTION

The present invention relates to barrier contraceptives, in particular to a condom holder.

### BACKGROUND ART

A condom is a skin made of natural latex or another material, covering the penis entirely or partially. Condoms are used to protect from sexually transmitted diseases and/or undesirable pregnancy. A traditional condom is shown in FIG. 1. A condom can also be a so-called micro-condom covering only the balanus.

To make it compact and easy to use, a condom is usually rolled into ring. Actually, it can also be rolled in an opposite direction to that traditionally used for rolling a condom. A condom can also be bellowed or otherwise folded. When stored, a condom is usually sealingly packed into a skin generally formed as a package of an elastic sheet material which is usually strong enough to provide tightness and protect the condom during storage and transportation (see FIG. 1). A package is usually provided with means facilitating its unpacking, e.g. a notch or reduced-strength area at its side, or a sealed ribbon with one its end extending outside the package. When unpacked, a condom is applied to erected penis and then rolled down to the penis root.

It can be unnecessary to unroll down a micro-condom because it covers only the balanus. Therefore, a micro-condom only requires a minimal degree of unrolling, if any.

The main disadvantage of traditional condoms is that it takes a long time and much effort to unpack and put on a condom. As a result, the user's attention is distracted away from sexual intercourse.

It is also necessary to use both hands to put the condom on. Typically, however, the user has only one free hand, and uses the other one to support himself or has it under woman's head (see FIG. 2). The movements of this hand are therefore restricted. Generally, this position is uncomfortable, and the user can release his second hand only for a short time. Therefore, for unpacking and putting on the condom, the user can use freely only one of his hands, occasionally using his second hand.

When putting on a condom, every moment spent can be critical for the user. Requirements for swift, convenient and easy handling of condom package are much higher than those for any other packed product used in private life. Besides, it is known that some people do not use condoms even when necessary, and the main reason for this is that they have to spend a long time and much effort to put a condom on, resulting in distraction of their minds from the sexual intercourse. The object is to simplify and reduce the number of steps required to unpack and put on a condom. If this problem is solved,

a) the overall condom usage will increase; whereas

b) the number of people who either do not use condoms at all, or use them rarely while having risky sexual intercourses, will decrease.

This could help to solve the spreading HIV problem as well as decrease the uncontrolled birth rate growth (especially in overpopulated developing countries).

Devices known as condom holders hold a condom, in most cases hold the edge of a rolled condom, and therefore allow the user grasp a condom holder and put the condom on by applying the condom on top of and down the penis. In this process, the condom is unrolled along the penis shaft (where a pre-rolled condom is used).

A condom holder can be a hollow ring with a groove extending along its circumference and defining a recess for receiving a condom. The shape and location of the groove can vary (e.g., see U.S. Pat. No. 4,738,357, Martin et al, 1988).

When the inner diameter of the holder is greater than or equal to the inner diameter of the condom ring, the recess can open to the outside. In this case, forces arise directed to the center of the condom ring, which do not let this ring pop out of the groove.

This kind of holder is disclosed in the following patents: JP6154260 (Toshihiro, 1994), JP7308336, Tadashi, 1995), U.S. Pat. No. 5,579,784 (Harari, 1996), DE19641107 (Raabe, 1998), U.S. Pat. No. 5,437,286 (Stratton, 1995), FR2751869 (Loyer, 1998).

Also known are condom holders either embracing the whole condom ring (e.g. see U.S. Pat. No. 5,163,449, van der Valk 1992) or having one discontinuity, either C-shaped or U-shaped (e.g. see U.S. Pat. No. 4,875,491, Parrone, 1989).

Furthermore, holders can either hold the condom ring with spaced fasteners (e.g. see FR2727858, Vegega, 1996) or continuously along the whole circumference of the ring.

Other means of holding the edge of a rolled condom can be used, e.g. the condom ring can be held by a pair of rollers (e.g. see WO9801095, Liehs, 1999).

However, mentioned holders touch the penis when putting on the condom, which is uncomfortable for the user. Thus, need for a holder that keeps the condom stretched while the fasteners do not touch or only slightly touch the penis arises.

A condom holder WO2007/021184 (Tunovic Adnan et al, 2007), is known that consists of two parts, each of said parts comprises at least one fastener formed as a number of nooses grasping the condom ring on the outer side. Due to the fact that penis contacts the fasteners through the condom, there is much less discomfort for the user than in case when the holder's fasteners embrace the condom ring on the inner side, thus contacting the penis directly. Nevertheless, the necessity to stretch the condom in order to reduce the pressure of the holder onto the penis remains.

FIG. 5 shows the holder consisting of two parts which are to be held with both hands (e.g. see WO02/069861, Van Rensburg, 2002). In this case, the user stretches the condom by bringing parts of the holder apart before putting the condom on. Then the user puts the condom on, while holding halves of the holder with both hands and displacing said holder towards the base of the penis. Therefore, in order to stretch the condom and subsequently put it on, the user is required to use both hands.

A holder that seems to be the closest to the one claimed herein is described in WO2006/025755 (Mor Maty Seck, 2006). The holder includes a ring, two opposite parts formed as semi-rings, and a radial guideway. One of the semi-rings is placed on said ring. A handle is inserted into the guideway, the handle is provided with another semi-ring on the side facing the ring. Both semi-rings are provided with a groove used to contain the condom edge rolled into a ring.

When putting on the condom held by holder, the ring with the guideway is held in one hand, while displacing the handle along the guideway, thus enlarging the inner area formed by two semi-rings and therefore stretching the condom ring to the required degree.

However, due to the condom ring being closed, the ring is also removed in longitudinal direction after putting the condom on, which is uncomfortable for the user.

Furthermore, stretching of the condom with such holder is performed using both hands, which is uncomfortable for the user due to aforementioned reasons.

Another disadvantage of said holder is lack of compactness thereof. Size of the ring is fixed and is defined by the size of a stretched condom. Furthermore, the size of the holder is extended due to the presence of the radial handle. Therefore, the size of the holder is significantly larger than the package of a conventional condom and is not quite suited for keeping in the user's pocket.

A method of putting on the condom that seems to be the closest to the one claimed herein is described in WO 2006/25755 (Mor Maty Seck, 2006). According to said method as described above, when putting on the condom held by holder, the ring with the guideway is held in one hand, while displacing the handle along the guideway, thus enlarging the inner area formed by two semi-rings and therefore stretching the condom ring to the required degree.

Therefore, in order to put the condom on, the user is required to use his second hand, which (as mentioned before) is uncomfortable and nullifies the potential advantage of said holder.

The purpose of this invention is to provide a durable and compact condom holder which would provide fast and comfortable stretching of the condom and, subsequently, fast and comfortable process of putting on the condom. It is preferrable that all actions performed while stretching and putting on the condom are performed using one hand, and it is possible to remove the holder in lateral direction after the condom is put on; therefore, the process of putting on the condom causes only minimal distraction of the user from sexual intercourse. It is also preferrable that the holder is foldable and takes as little space as possible in the user's pocket.

### BRIEF SUMMARY OF THE INVENTION

The problem is solved by providing a condom holder at least partially defining a space in which the flatly arranged condom can be placed, that comprises fasteners used for fixing the condom edge; a connection between said fasteners that includes two opposite parts, each of which includes at least one of said fasteners; a third part placed between said parts that provides the moveability of said opposite parts at least in opposite directions; and a device used to displace said opposite parts using the mechanical force applied by the user; said third part is formed in such manner that said moveability of opposite parts in opposite directions includes the possibility of rotation thereof.

Thus, only one hand is required to stretch the condom and subsequently put it on using simple motion of the digits of said hand. Due to the fact that the holder comprises two opposite parts adapted to displace in opposite directions, and the third part providing said displacement, the process of stretching the condom is fast and convenient.

The holder can be provided with a holder locking device for locking the holder in displaced state.

Each of the opposite parts can include at least one band.

The band can be adapted to bend inside the area defined by the holder.

Furthermore, the band can be provided with a stopper restricting the bending inside the area defined by the holder.

The bending ability can be provided by an elastic connector or a hinge joint.

Said rotation ability can be provided by an elastic connector or a hinge joint.

Said third part can include an elastic connector or a hinge joint.

The holder can include two levers, with one of the arms formed by said opposite parts and the other arm formed by said displacement device.

The third part can include two transfer members, each of which is hinged to one of the opposite parts; two transfer members are rotatably connected to each other.

The fasteners can be noose-shaped and adapted to hold the edge of a rolled condom on the outer side of said condom.

Furthermore, the problem is solved by providing a condom holder at least partially defining a space in which the flatly arranged condom can be placed, that comprises two levers, first arm of which includes at least one fastener for fixing the condom edge, and said levers are connected to each other and adapted to displace first arms thereof at least in opposite directions, while two other arms thereof are adapted to be affected mechanically by the user in order to provide said displacement.

The holder can be provided with a holder locking device for locking the holder in displaced state.

Said levers can possess a common pivot at which said levers are connected.

Said levers can be connected to each other with a connector, and the pivots thereof are located on the ends of said connector.

The levers can be adapted to be affected by the digits of one hand in the condom plane.

Arms of the levers adapted to be affected mechanically by the user are rotatably connected to the first arms and the ability to be placed (?) along said first arms.

The holder can be received in a box open at one end and including a slot used as a guideway; the holder can be provided with a rest connected to said holder by a part moveably placed in said slot; the rest is placed outside the box and arms of the levers adapted to be affected mechanically by the user are rotatably connected to the box.

Each lever can include at least one band.

The band can be adapted to bend inside the area defined by the holder. Furthermore, the band is provided with a stopper restricting the bending inside the area defined by the holder.

The bending ability is provided by an elastic connector or a hinge joint.

Furthermore, the problem is solved by providing a condom holder at least partially defining a space in which the flatly arranged condom can be placed, that comprises two single-arm levers, first section of which includes at least one fastener for fixing the condom edge, and said levers are connected to each other and adapted to displace first sections of the arms thereof at least in opposite directions, and each lever includes another section adapted to be affected mechanically by the user in order to provide said displacement.

The holder can be provided with a holder locking device for locking the holder in displaced state.

Said levers can intersect in such manner that the section of each lever adapted to be affected mechanically by the user is placed sidewise to the other lever.

In one modification, sections of the levers adapted to be affected mechanically by the user can be placed closer to the pivot than the first sections; in another modification, the first sections of the levers can be placed closer to the pivot than sections of the levers adapted to be affected mechanically by the user.

Said levers can possess a common pivot at which said levers are connected.

Each lever can include at least one band.

The band can be adapted to bend inside the area defined by the holder. Furthermore, the band can be provided with a stopper restricting the bending inside the area defined by the holder.

The bending ability can be provided by an elastic connector or a hinge joint.

Furthermore, the problem is solved by providing a condom holder at least partially defining a space in which the flatly arranged condom can be placed, that comprises two opposite parts, each of which includes at least one fastener used for fixing the condom edge; said opposite parts are connected to each other and adapted to displace in at least opposite directions; each of said parts includes a transfer member hinged to said part, and two transfer members of two parts of the holder are rotatably connected to each other.

The holder can be provided with a locking device for locking said opposite parts in displaced state.

In one modification, said transfer members can be hinged to each other.

In another modification, said transfer members can be hinged to the end of a rod adapted to displace along the guideway which is also used to connect parts of the holder to each other due to the fact that said parts of the holder are hinged to said guideway by ends of said parts.

The holder can be received in a box open at one end and including a slot used as a guideway; the holder is provided with a rest connected to the end of the rod by a part moveably placed in said slot; the rest can be placed outside the box.

Said opposite parts can include at least one band.

The band can be adapted to bend inside the area defined by the holder.

Furthermore, the band can be provided with a stopper restricting the bending inside the area defined by the holder.

Furthermore, the problem is solved by providing a condom holder at least partially defining a space in which the flatly arranged condom can be placed, that comprises two angular parts connected moveably to each other and arranged in such manner that that first sections thereof, each including at least one fastener for holding the condom edge are placed opposite with respect to each other, and their second sections, angled with respect to the first sections, extend along each other, said connection of the angular parts is arranged at second sections thereof, providing said displacement of the angular parts by displacing second sections thereof along each other with corresponding displacement of first sections thereof at least in opposite directions.

The holder can be provided with a holder locking device for locking the angular parts in displaced state.

Said angular parts can include corresponding handles on the ends of second sections thereof; said handles are used to provide the convenient process of affecting said angular parts mechanically in order to displace said parts.

Said handles can be connected to the angular parts with ability to rotate and to be placed along the angular parts.

Said handles can be adapted to be placed along the first sections.

Said angular parts can intersect in such manner that the handle of each angular part is placed sidewise to the other angular part.

Said second sections can be located in one plane or one above the other.

The holder can include a guideway with an opening embracing the second sections.

Each angular part can include at least one band.

The band can be adapted to bend inside the area defined by the holder. Furthermore, the band can be provided with a stopper restricting the bending inside the area defined by the holder.

The bending ability can be provided by an elastic connector.

The bending ability can be provided by a hinge joint.

Furthermore, the problem is solved by providing a method of putting on the flatly arranged condom held by a holder at least partially defining the area in which the condom is placed and including two opposite parts, each of which includes at least one fastener for holding the condom edge and a device used to displace said opposite parts using the mechanical force applied by the user, according to which the holder is held in a hand and said opposite parts are displaced till the condom is stretched, then the condom is put on by displacing the holder towards the base of the penis; said displacement is performed by affecting said opposite parts with at least two digits of the same hand holding the holder.

Furthermore, said opposite parts are locked in displaced state.

The locking is performed with a digit of the same hand holding the holder.

The affection can be pressing said opposite parts in the condom plane.

The pressing is performed by at least the thumb and the index finger of the hand holding the holder.

The displacement is performed towards the palm of a hand or in the opposite direction.

The levers formed by said opposite parts are affected.

The first arms of said levers are displaced in opposite directions by affecting the second arms of the levers with two digits of the same hand by displacing said arms towards each other.

The levers can be single-arm.

The first sections of the arms of said levers are displaced in opposite directions by affecting the second sections of the arms of the levers placed closer to the pivot than first sections by displacing second sections of said arms towards each other.

The first sections of the angular parts of the holder formed by said opposite parts are displaced in opposite directions by affecting the second sections of the angular parts with two digits of the same hand by displacing said sections along each other.

Said affection of the second sections of the angular parts is performed by affecting the handles placed on the ends thereof.

Said displacement of the opposite parts is performed by affecting the connection point of transfer members by a digit of the same hand.

The base connected to the holder and adapted to be held in hand is held, while the holder is displaced by affecting the connection point of transfer members with a digit of the same hand holding the base.

Furthermore, the problem is solved by providing a condom holder at least partially defining a space in which the flatly arranged condom can be placed, that comprises fasteners used for fixing the condom edge; a connection between said fasteners that includes two opposite parts connected to each other by displacement device providing moveability thereof at least in opposite directions, each of said opposite parts includes at least one of said fasteners; and a locking device locking the holder in displaced state, said displacement device is formed in such manner that said moveability of opposite parts in opposite directions includes the possibility of rotation thereof.

Each opposite part can include a band, with one end thereof connected to displacement means, and the other end is free.

The displacement means can include a hinge joint.

Furthermore, the problem is solved by providing a method of putting on the flatly arranged condom held by a holder at least partially defining the area in which the condom is placed and including two opposite parts, each of which includes at least one fastener for holding the condom edge, a displacement device placed between said parts and providing moveability thereof using the mechanical force applied by the user, and a locking device keeping the holder in displaced state, according to which the user holds the holder in both hands, displaces said opposite parts till the locking device locks the opposite parts in displaced state, then the user takes the holder in one hand, puts the condom on by displacing the holder towards the penis root, and removes the holder; said opposite parts are rotated with respect to each other in the condom plane, and the holder is removed in transverse direction.

### BRIEF DESCRIPTION OF THE DRAWINGS

Below is a detailed description of the invention with references to the attached drawings, in which:

FIG. 1 is a perspective view of the traditional condom held in hand by the user;

FIG. 2 illustrates the situation preceding the process of putting on the condom;

FIG. 3 is a perspective view of the condom placed in a known holder consisting of two parts held by the user;

FIG. 4 is the perspective view of the condom holder in one of its embodiments;

FIG. 5 is a perspective view of the condom placed in the holder represented in FIG. 4;

FIG. 6 is a perspective view with an enlarged portion brought out of the condom placed in the holder represented in FIG. 5, with the condom stretched.

FIG. 7 is a perspective view of another embodiment of the condom holder;

FIG. 8 is a perspective view of the condom holder shown in FIG. 7, unfolded;

FIG. 9 is a perspective view of another embodiment of the condom holder;

FIG. 10 is a perspective view of the condom holder shown in FIG. 9, unfolded;

FIG. 11 is a perspective view of another embodiment of the condom holder;

FIG. 12-13 are perspective views of the unfolding of the holder shown in FIG. 11;

FIG. 14 is a perspective view of another embodiment of the condom holder;

FIG. 15 is a perspective view of the condom holder shown in FIG. 14, unfolded;

FIG. 16 is a perspective view of another embodiment of the condom holder, received in a package;

FIG. 17 is a perspective view of another embodiment of the condom holder;

FIG. 18 is an exploded view of the parts of the holder shown in FIG. 17;

FIG. 19 is an exploded view of the parts of the holder package shown in FIG. 17;

FIGS. 20-23 are perspective views of the process of unpacking the condom held by the holder shown in FIG. 17;

FIG. 24 is a perspective view of another embodiment of the condom holder;

FIG. 25 is a perspective view of the condom holder shown in FIG. 24, unfolded;

FIG. 26 is a perspective view of another embodiment of the condom holder;

FIG. 27 is a perspective view of the condom holder shown in FIG. 26, unfolded;

FIG. 28 is a perspective view of another embodiment of the condom holder;

FIG. 29 is a perspective view of the condom holder shown in FIG. 28, unfolded;

FIG. 30 is a perspective view of another embodiment of the condom holder with a condom therein;

FIG. 31 is a perspective view of the holder with a condom therein shown in FIG. 30, unfolded;

FIG. 32 is a perspective view of another embodiment of the condom holder with a condom therein;

FIG. 33-35 are perspective views of the process of unpacking the condom held by the holder shown in FIG. 32;

FIG. 36 is a schematical top view of another embodiment of the holder;

FIGS. 37-39 are schematical top views of the process of unpacking the condom held by the holder shown in FIG. 36;

FIG. 40 is a perspective view of another embodiment of the condom holder;

FIG. 41 is a perspective view of the condom holder shown in FIG. 40, unfolded;

FIG. 42 is a perspective view of another embodiment of the condom holder;

FIG. 43 is a perspective view of the condom holder shown in FIG. 42, unfolded;

FIG. 44 is a perspective view of another embodiment of the condom holder;

FIG. 45 is a perspective view of the condom holder shown in FIG. 44, unfolded;

FIG. 46 is the perspective view of the suggested condom holder in its another embodiment;

FIG. 47 is an exploded view of the parts of the condom holder shown in FIG. 46;

FIG. 48 is a perspective view of another embodiment of the condom holder;

FIG. 49 is a perspective view of the device for combined unpacking and unfolding of a condom holder;

FIG. 50 is a perspective view of the condom holder shown in FIG. 49;

FIGS. 51-52 are perspective views of the assemblage stages of the device shown in FIG. 49;

FIGS. 53-54 are perspective views of the unpacking and unfolding stages of the holder shown in FIG. 50;

FIG. 55 is the perspective view of the suggested holder in its another embodiment;

FIG. 56 is a perspective view of the holder with a condom therein shown in FIG. 55, unfolded;

FIG. 57 is a perspective view of another embodiment of the condom holder;

FIG. 58 is a perspective view of the holder shown in FIG. 57, unfolded;

FIGS. 59-61 illustrate the process of putting on a condom with the holder shown in FIG. 57.

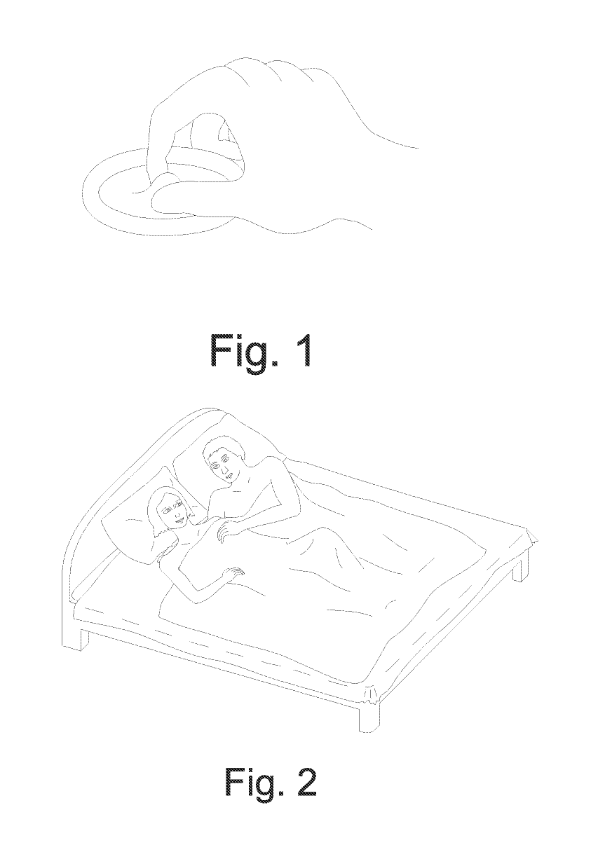

### DETAILED DESCRIPTION OF THE INVENTION

In FIGS. 4-6, one embodiment of the invention is represented. In this embodiment, a holder 1 includes two arc-shaped bands 2 and 3 connected to each other by a flexible connector 4. Bands 2 and 3 connect fasteners 5 holding the condom; said fasteners are placed on the inner side 6 of the bands 2 and 3.

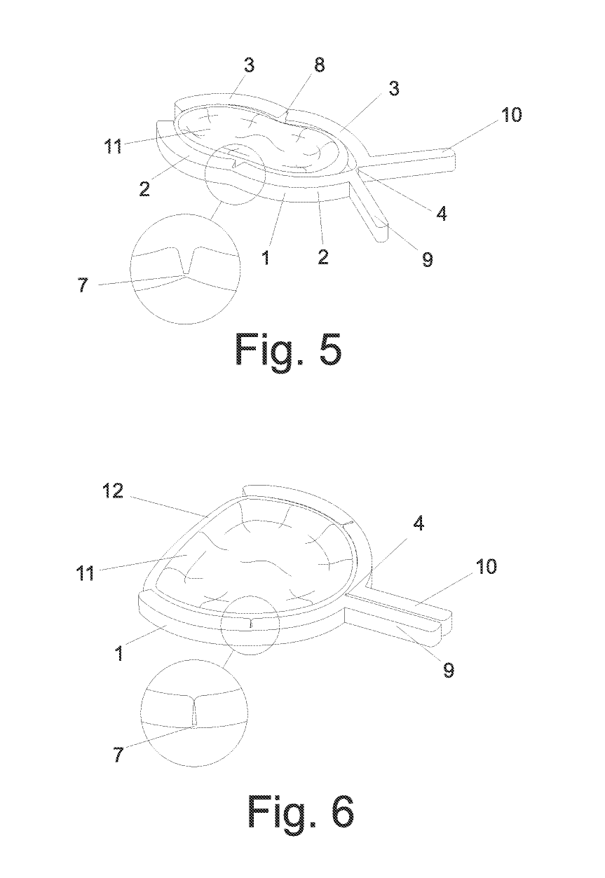

As shown in FIGS. 4 and 5, the holder includes two flexible connectors 7 and 8 formed on the inner side of the bands 2 and 3. Said connectors 7 and 8 allow to keep the holder with the condom therein in a folded state, as shown in FIG. 5. This, in turn, provides the possibility of keeping the condom in unstretched state and also reduce the size of the package in which the holder is received, as shown in FIG. 5.

Handles 9 and 10 are attached to bands 2 and 3 on the outer side 13 thereof; said handles can be used for holding the holder when putting the condom on. Said handles 9 and 10 are placed in close proximity to the connector 4 on two sides thereof.

The band 2 with the handle 9, and the band 3 with the handle 10 form the levers with the common rotation point thereof placed on the connector 4.

A condom 11 is placed into the holder 1, as shown in FIGS. 5 and 6, and the rolled edge 12 of the condom is placed in the fasteners 5.

The device is operated as follows: The user takes the device in his hand, holding it by handles 9 and 10 with two digits. Then he affects the handles, displacing them towards each other. The area defined by the holder 1 is therefore expanded, and the condom 11 is stretched. The mentioned holder design allows the user to stretch the condom easily with one hand by performing a single action: moving two digits of a hand towards each other.

Further, the holder 1 is held by the handles 9 and 10 and displaced towards the base of the penis (not shown); afterwards, the holder 1 is removed in transverse direction. The above is possible due to the fact that the holder is open and it embraces the penis less than for 180°.

Therefore, for performing all actions required to put on the condom, the user can use only one hand.

Connectors similar to connectors 7 and 8 shown in FIG. 5, can also be placed on the outer side of the holder. In this case, the holder is additionally provided with flexible bands attached to said holder on the edges of said connectors and restricting the rotation of bands 2 near (?) said connectors.

Furthermore, instead of the connector 4 shown in FIG. 5, a hinge joint can be used.

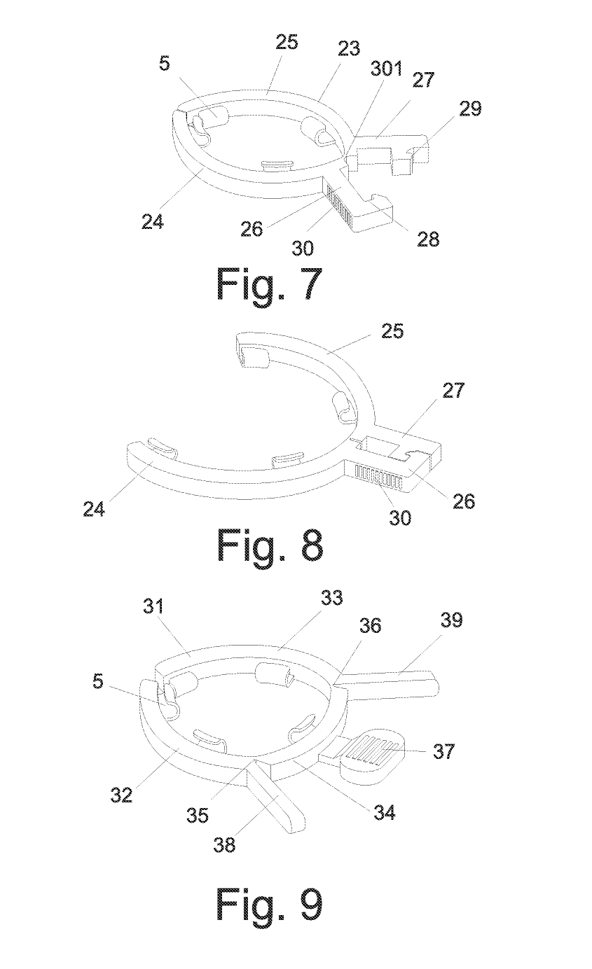

In the embodiment represented in FIGS. 7-8, a holder 23 includes two arc-shaped bands 24 and 25 connected to each other by a flexible connector 301. Fasteners 5 are placed on the inner side of said bands.

Handles 26 and 27 are placed in close vicinity to the connector 301 and are attached to bands 24 and 25 on two sides thereof.

The handle 26 is provided with a slot 28 on the end thereof, and the handle 27 is provided with a lug 29 adapted to mechanically engage with the slot 28. By means of said slot 28 and said lug 29, handles 26 and 28 can be locked with respect to each other, and thus the holder 23 can be locked in unfolded state (FIG. 8). Each handle 26, 27 is provided with ribbed sections 30 providing the mechanical engagement of the digits with the surface thereof (FIG. 8). The mentioned handle design makes the process of putting on the condom more convenient.

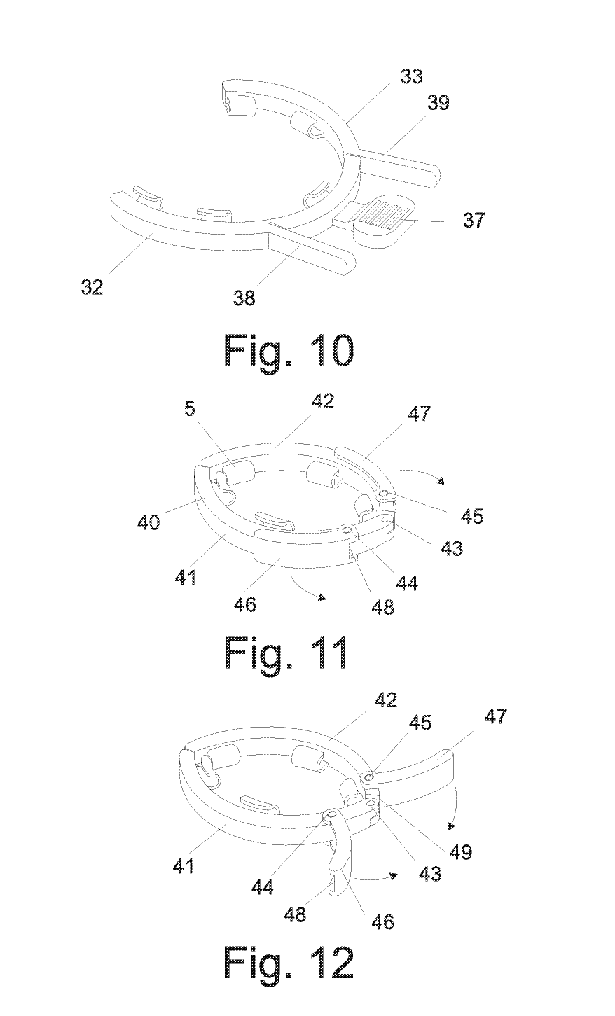

In yet another embodiment represented in FIGS. 9-10, a holder 31 includes three arc-shaped bands 32, 33 and 34 connecting the fasteners 5 to each other. Bands 32 and 34 are connected to each other by a flexible connector 35, and bands 33 and 34 are connected to each other by a flexible connector 36. A handle 37 is attached to the band 34 on the outer side thereof; said handle can be used for holding the holder when putting the condom on. Handles 38 and 39 are placed in close vicinity to the connectors 35 and 36 and are attached to bands 32 and 33, as shown in FIG. 9. The band 32 with the handle 38, and the band 33 with the handle 39 form the levers with the rotation points thereof placed on the connectors 35 and 36 respectively.

The process of putting on the condom held by the holder 31 is similar to that disclosed for the holder 1 shown in FIGS. 4-6.

In the embodiment represented in FIGS. 11-13, a holder 40 includes two arc-shaped bands 41 and 42 connected to each other by a hinge joint 43. Fasteners 5 are placed on the inner side of said bands.

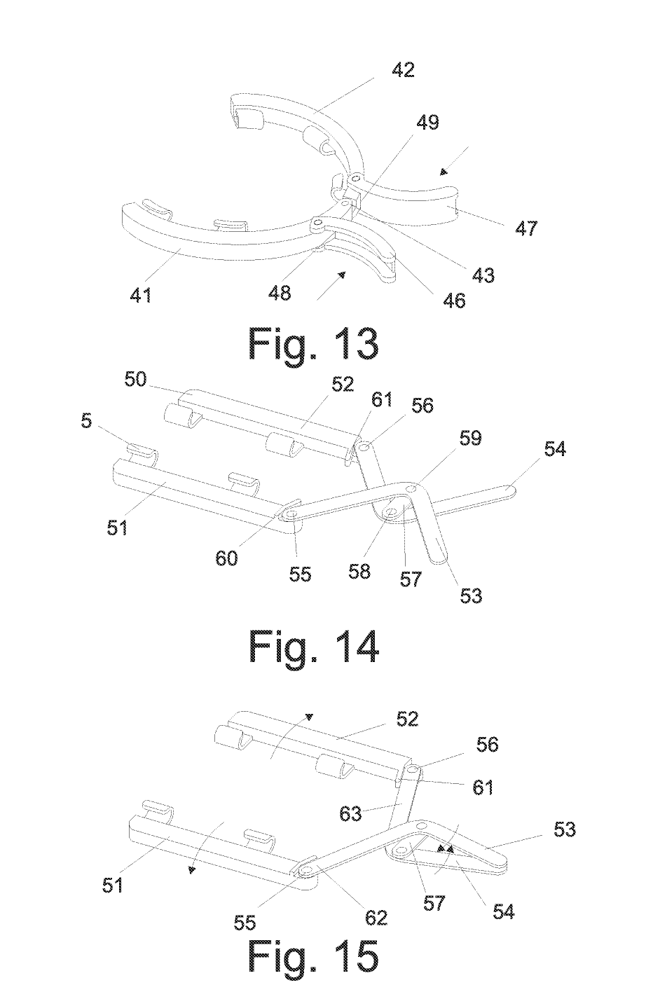

Handles 46 and 47 are placed in close vicinity to the hinge joint 43 and are attached to bands 41 and 42 on two sides of said hinge joint by additional hinge joints 44 and 45. Handles 46 and 47 are arc-shaped and possess a U-shaped profile facing the bands 41 and 42 with the inner side thereof. Handles 46 and 47 embrace bands 41 and 42 when folded, as shown in FIG. 11. Cutouts 48 and 49 are located on the end by which handles 46 and 47 are connected to bands 41 and 42; said cutouts provide the possibility of rotating said handles up to a defined position shown in FIG. 12. The mentioned design of handles 46 and 47 allows a compact holder design.

The band 41 with the handle 46, and the band 42 with the handle 47 form the levers with the common rotation point thereof matching the hinge joint 43.

Prior to putting on the condom (not shown) held by the holder 40, the handles 46 and 47 are rotated in a direction shown with arrows in FIGS. 11-12, until the cutouts 48 and 49 rest upon the bands 41 and 42. When affecting the handles 46 and 47 further in a direction shown with arrows in FIG. 12, the force applied to said handles transfers to bands 41 and 42, which leads to said bands starting to displace in opposite directions as shown in FIG. 13. The condom (not shown) is therefore stretched and ready to be put on. Further, the holder 40 is displaced towards the base of the penis (not shown), holding said holder with a digit of a hand by the handles 46 and 47. Afterwards, the holder is removed in transverse direction.

A holder 50 shown in FIGS. 14-15, includes two straight bands 51 and 52, each connecting the fasteners 5 to each other. L-shaped handles 53 and 54 are attached to bands 51 and 52 on one end thereof by hinge joints 55 and 56. Said handles 53 and 54 are attached to the ends of a strip 57 by hinge joints 58 and 59. Bands 51 and 52 are provided with lugs 60 and 61 placed on the same end thereof, on which hinge joints 55 and 56 are formed. Said lugs restrict the rotation of bands 51 and 52 with respect to handles 53 and 54 around the hinge joints 55 and 56 up to a defined position. The band 51 with the handle 54, and the band 52 with the handle 53 form the levers with the pivots thereof placed on the rotation axes of hinge joints 58 and 59 respectively.

Prior to putting on the condom (not shown) held by the holder 50, the user affects the ends 62 and 63 of L-shaped handles 53 and 54, displacing said handles towards each other until said handles rest upon the lugs 60 and 61.

When affecting the handles 53 and 54 further, the force applied to said handles transfers to bands 51 and 52, which leads to said bands starting to displace in opposite directions as shown in FIG. 15. The area defined by the holder is expanded, and the condom is stretched. The force applied to handles 53 and 54 is compensated by the compressive forces of the condom edge.

An embodiment is possible, where a condom held by holder is packed, and the unpacking of said condom is combined with stretching thereof.

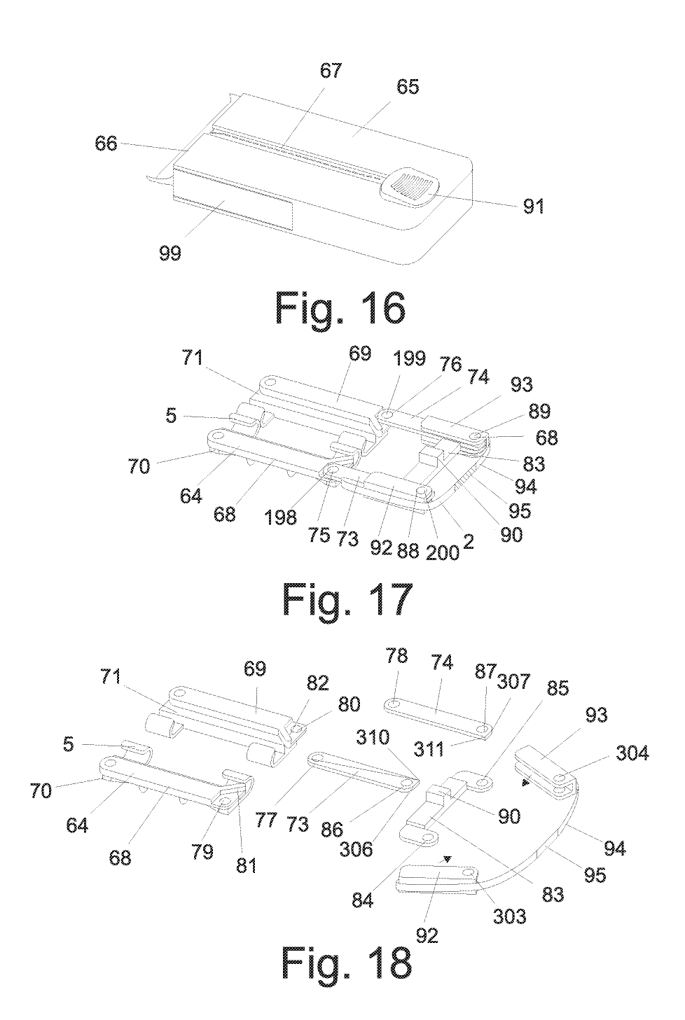

For example, as shown in FIGS. 16-23, a holder 64 is received in a flat box 65, which includes one open end 66 and a central through slot 67 formed on one side of the box 65 (FIG. 16). The box 65 can be made of any material possessing sufficient strength to provide the integrity of the contents and the possibility of holding it in hand, e.g. it can be made of plastic.

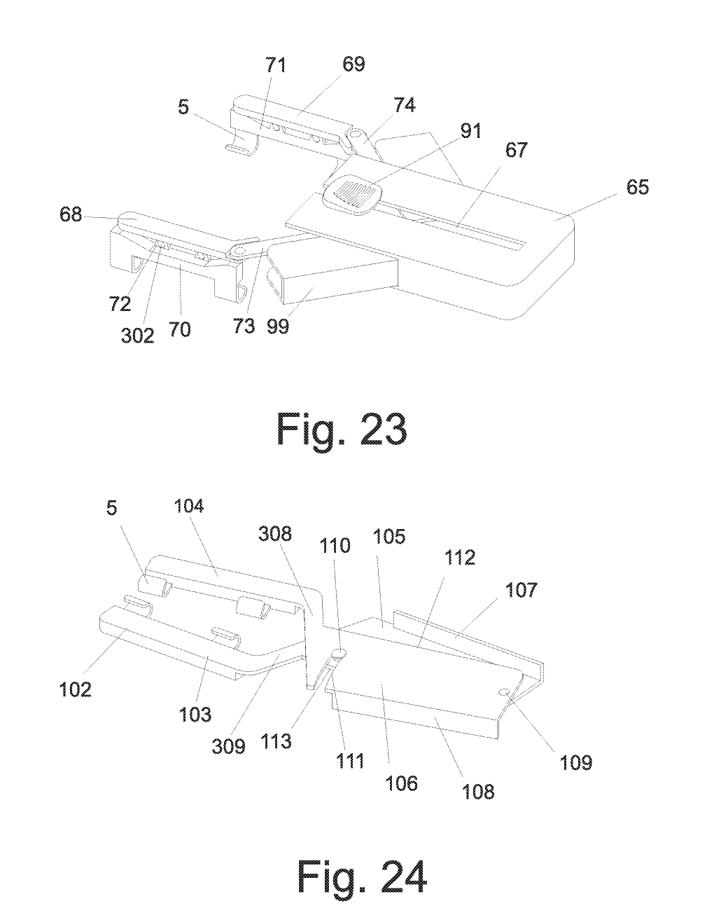

As shown in FIGS. 17, 18, the holder 64 includes bands 68 and 69. Fasteners 5 are connected pairwise by strips 70 and 71 (FIG. 23), each of which includes ears 72 connected to nooses 302 attached to bands 68 and 69, and adapted to rotate. Such connection of strips 70 and 71 to the bands 68 and 69 provides the rotation of said strips and, consequently, of fasteners 5 in a direction opposite to that in which the hooks of the fasteners are bent.

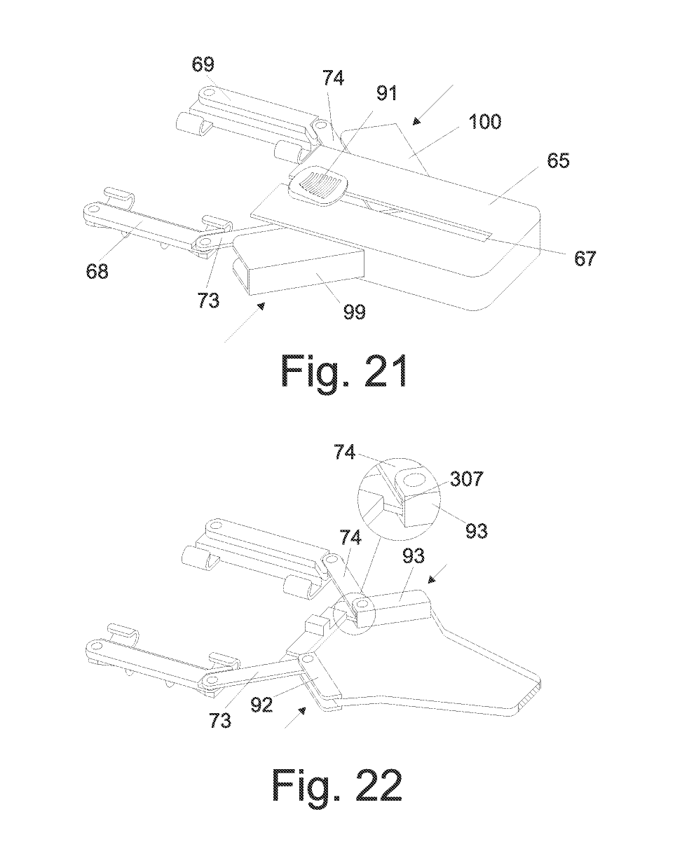

Bands 73 and 74 are connected to bands 68 and 69 by hinge joints 75 and 76 (FIGS. 17, 18). For this, bands 73 and 74 are provided with openings 77 and 78 on one end thereof, and bands 68 and 69 are provided with openings 79 and 80; said openings are placed on axes 198 and 199, thus forming hinge joints 75 and 76 respectively. Lugs 81 and 82 are formed on bands 68 and 69 in close vicinity to the openings 79 and 80; said lugs restrict the rotation of bands 73 and 74 up to a defined position shown in FIGS. 21-23.

A strip 83 is placed between bands 73 and 74 and includes openings 84 and 85 on the ends thereof; said openings are connected to openings 86 and 87 formed on bands 73 and 74 by axes 200 and 201. Openings 84 and 86, 85 and 87 (FIG. 18) and said axes form hinge joints 88 and 89 (FIG. 17). A rest support 90 is formed in the middle part of the strip 83; said rest support is adapted to rotate in the slot 67 when placing the holder 64 in the box 65, and a rest 91 is attached to said rest support on the outer side of the box (FIG. 16).

Levers 92 and 93 provided with openings 303 and 304 placed on the hinge joint axes 200 and 201, are also attached to hinge joints 88 and 89. Levers 92 and 93 possess a U-shaped profile and are facing the bands 73 and 74 with the inner side thereof, embracing said bands. Ends 306 and 307 of the bands 73 and 74 include sharp corners 310 and 311; said corners restrict the rotation of said bands with respect to levers 92 and 93, as shown enlarged in FIGS. 18, 22. A flexible ribbon 94 connecting levers 92 and 93 to each other embraces levers 92 and 93 on the outer side thereof, and is attached (e.g. glued) to said levers on the inner side thereof. The ribbon 94 includes a section 95 attached to the box 65 on the inner side of the closed end thereof when placing the holder 64 in said box.

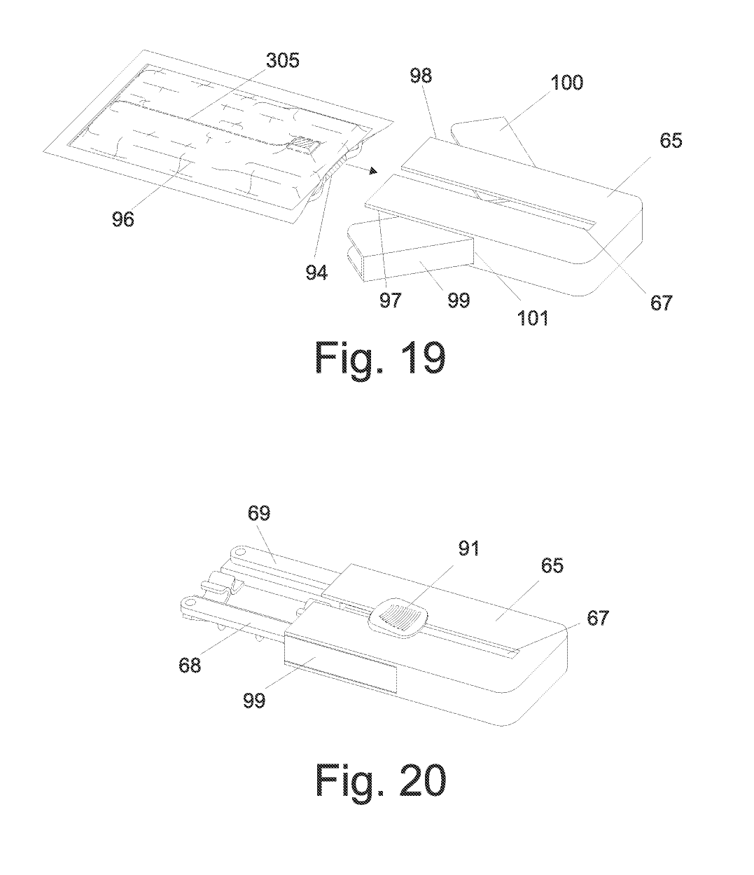

The holder 64 is received in a package 96 similar to a conventional condom package; the ribbon 94 is placed outside the package 96. Package 96 can be provided with weakened lines 305, along which it can be easily torn. The preferrable arrangement of weakened lines is shown in FIG. 19.

The package 96 is bonded to the box 65, e.g. it can be glued. The attachment points of the package 96 to the box 65 are preferrably arranged on the end of the package located in close vicinity to the closed end of the box. However, the package 96 can be attached at longer areas.

The box 65 is provided with cutouts 97 and 98 formed on the open end 66; keys 99 and 100 are placed in said cutouts; said keys possess a U-shaped profile and are faced towards the box 65 with inner side thereof. Keys 99 and 100 are attached to the box 65 by flexible connectors 101.

The assembled device is shown in FIG. 16.

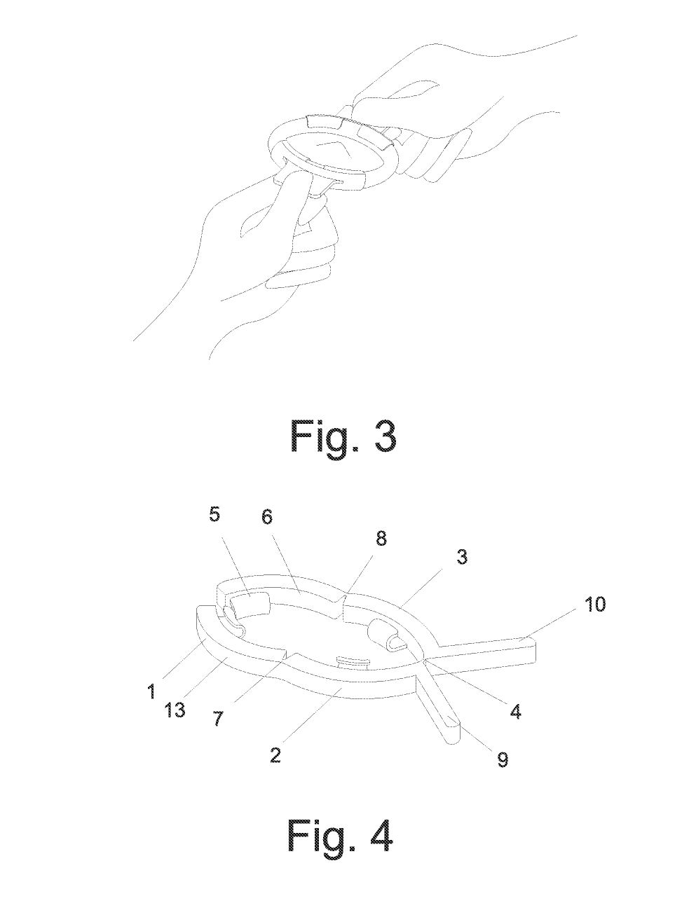

The device is operated as follows: The user holds the device in hand in such manner that the open end 65 of the box is faced forward, away from the user, and sets his thumb on the rest 91. Then the user moves said thumb forward, away from the user. The holder 64 is therefore displaced, tears the package 96 and comes out. When the rest 91 reaches the end of the slot 67, bands 68 and 69 leave the box 65 completely. Furthermore, due to the fact that the ribbon 94 is attached to the box 65, said ribbon will restrain levers 92 and 93, and said levers will start to rotate outwards; subsequently, due to the fact that keys 99 and 100 adjoin levers 92 and 93, said keys will move apart, as shown in FIGS. 21-3. Levers 92 and 93 rotate with respect to bands 73 and 74 until corners 310 and 311 of the ends 306 and 307 rest upon the inner surface of levers 92 and 93. Bands 73 and 74 are therefore fixed at a certain angle with respect to levers 92 and 93. Affecting levers 92 and 93 further leads to the unfolding of bands 73 and 74 connected to said levers, and to the unfolding of bands 68 and 69 stretching the condom (not shown).

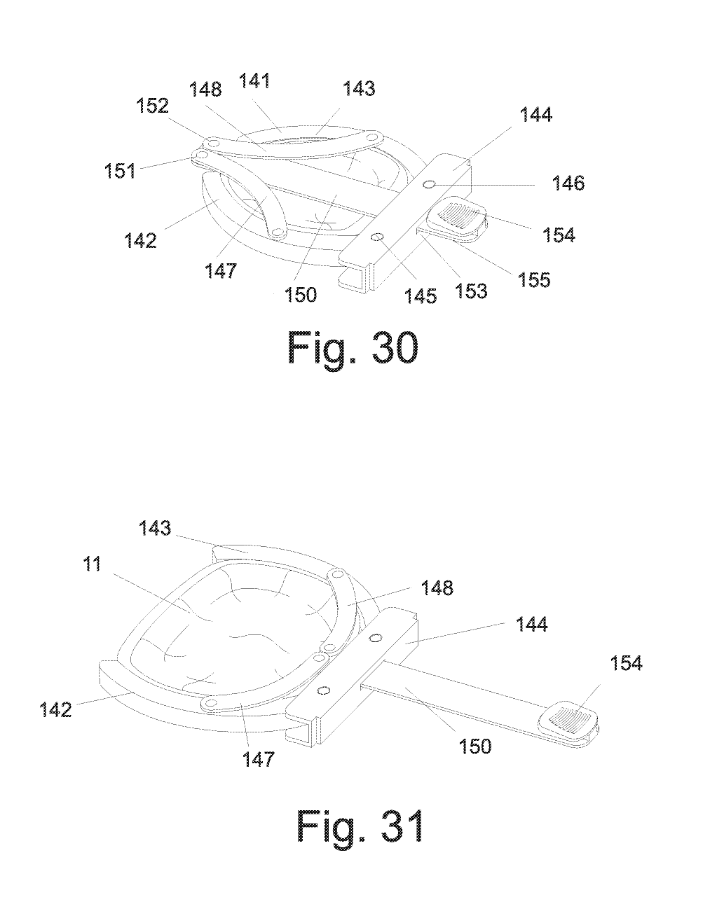

After the condom is stretched, the user puts it on, displacing the holder 64 towards the base of the penis. Afterwards, the user presses the keys 99 and 100 thus displacing levers 92 and 93 towards each other. Subsequently, the displacement of levers 92 and 93 leads to displacement of bands 73 and 74 and bands 68 and 69 towards each other; the fasteners 5 are pressed to the penis. Affecting the keys 99 and 100 further leads to the rotation of fasteners 5 in a direction opposite to that in which the hooks of fasteners are bent, as shown in FIG. 30. The condom edge is released from the fasteners, and the holder can be easily removed in transverse direction.

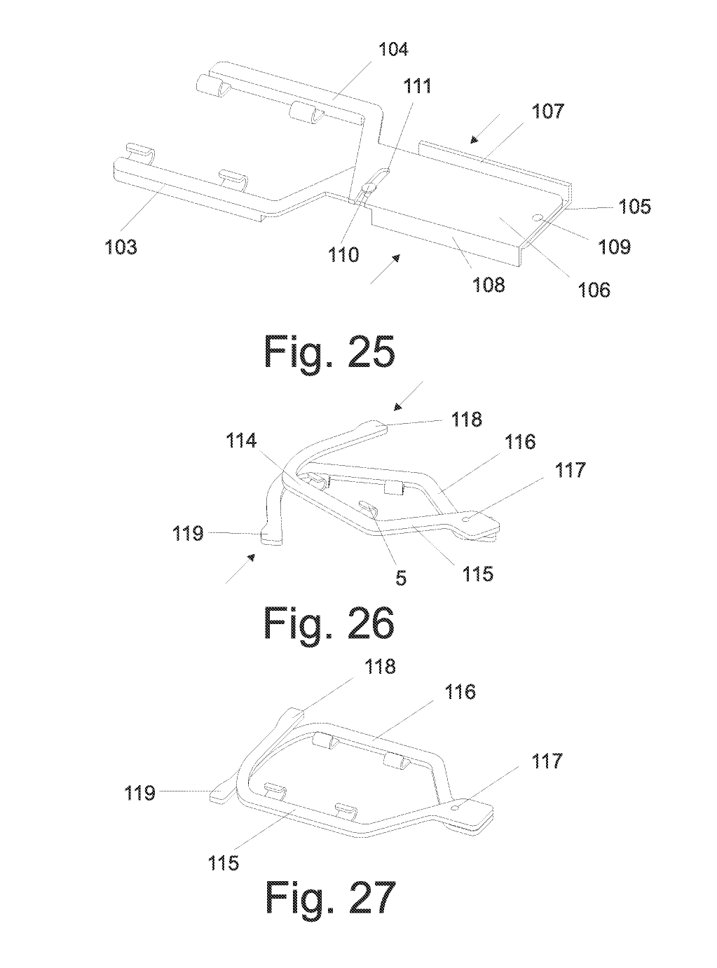

In FIGS. 24-25, yet another invention embodiment is represented. A holder 102 includes two straight bands 103 and 104 with fasteners 5 are attached to said bands. Plates 105 and 106 are attached to said bands 103 and 104 on the ends thereof. The plate 105 includes a flange 107, and the plate 106 includes a flange 108; said flanges are perpendicular to said plates. Flanges 107 and 108 serve as a support for the digits. Plates 105 and 106 are arranged one upon the other and connected to each other by a hinge joint 109. Thus, the plate 105 includes a lug 110 on the opposite side, and the plate 106 includes a slot 111 used as a guideway for the lug 110.

Connecting members 308 and 309 connect bands 103, 104 and bands 105 and 106, and are arranged crosswise.

Prior to putting on the condom, the user takes holder 102 in his hand, holding it by flanges 107 and 108. Afterwards, he affects said flanges with digits of the hand in a direction shown with arrows in FIG. 25. The lug 110 is therefore displaced in the slot 111. The action is performed until the lateral side 112 of the plate 106 rests upon the flange 107 of the plate 105, and the lateral side 113 of the plate 105 rests upon the flange 108; the area defined by the holder 102 is therefore expanded, and the condom (not shown) is stretched. Then the user puts the condom on, holding the holder 102 in unfolded state and displacing it towards the base of the penis. After the condom is put on, the holder can be removed in transverse direction.

A holder 114 shown in FIGS. 26-27, is formed by two curvilineal bands 115 and 116, each connecting the fasteners 5 to each other. Said bands 115 and 116 are connected to each other on one end by a hinge joint 117. The bands are arranged in such manner that in the home position, the other ends 118 and 119 of the bands 115 and 116 intersect and are faced in opposite directions as shown in FIG. 26.

Prior to putting on the condom (not shown), the user holds the ends 118 and 119 and displaces said ends towards each other, as shown with arrows in FIG. 26. The area defined by the holder 114 is therefore expanded, as shown in FIG. 27.

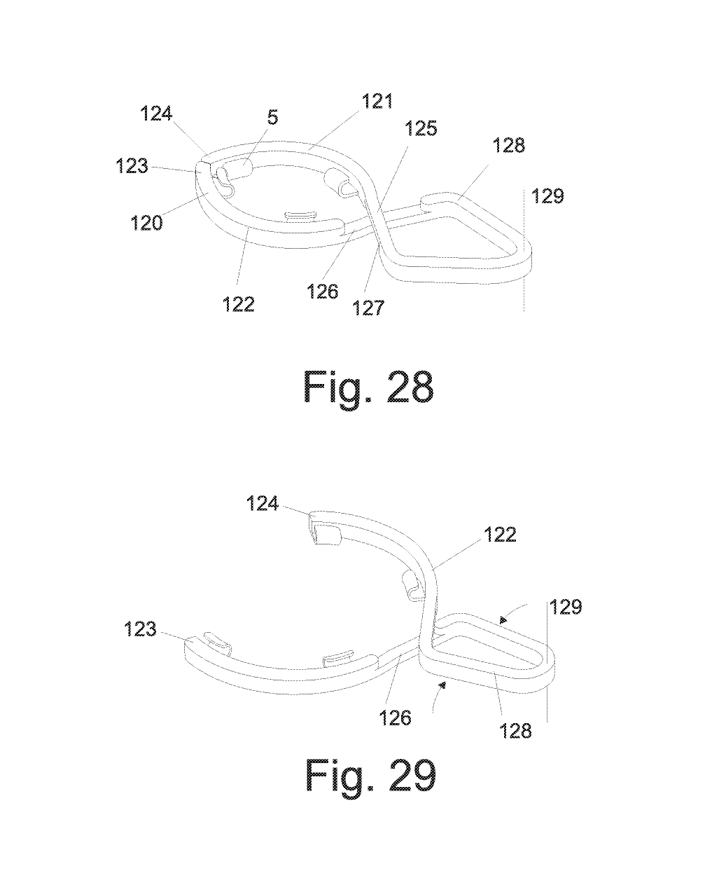

In yet another embodiment represented in FIGS. 28-29, a holder 120 includes a band 121 shaped as an open figure of eight, which connects the fasteners 5 to each other. Said fasteners are placed on the open part 122 of the holder between ends 123 and 124 and a junction 125 of the band 121. The band 121 includes two cutouts 126 and 127 formed in the vicinity of junction 125 facing each other. The mentioned design of cutouts 126 and 127 allows to arrange fasteners 5 in one plane. The closed part 128 of the holder 120 is made of an elastic material and can be arched around an axis 129. Therefore, the holder 120 is a system of two single-arm levers with a common rotation point placed on said axis 129.

Prior to putting on the condom, the user presses the closed part 128 of the holder with two digits in a direction shown with arrows in FIG. 29; ends 123 and 124 are therefore displaced in opposite directions, the area defined by the open part 122 of the holder is expanded (FIG. 29), and the condom (not shown) is stretched. Then the user puts the condom on, holding the holder 120 in unfolded state and displacing it towards the base of the penis.

Yet another embodiment of a compact holder is represented in FIGS. 30-31. A holder 141 includes arc-shaped bands 142 and 143 connecting the fasteners to each other. Said bands 142 and 143 are attached on one end to a frame 144 by hinge joints 145 and 146. The frame 144 possesses a U-shaped profile and is facing the ends of bands 142 and 143 with the inner side thereof, embracing said ends. The holder 141 includes transfer members formed as mainly arc-shaped strips 147 and 148. Strips 147 and 148 are hinged to bands 142 and 143 on one end, and attached to a straight rod 150 on the other end by two hinge joints 151 and 152. The rod 150 passes through an opening 153 formed in the frame 144, and is provided with a rest 154 on the end 155 thereof.

The device is operated as follows: The user holds the holder 141 in hand and displaces the rod 150 towards himself by affecting the rest 153. Strips 147 and 148 are therefore rotated and displace bands 142 and 143 in opposite directions, as shown in FIG. 31. When the rod is completely pushed out, the condom 11 is stretched and ready to be put on. Then the user puts the condom on, holding the holder 141 in unfolded state and displacing it towards the base of the penis.

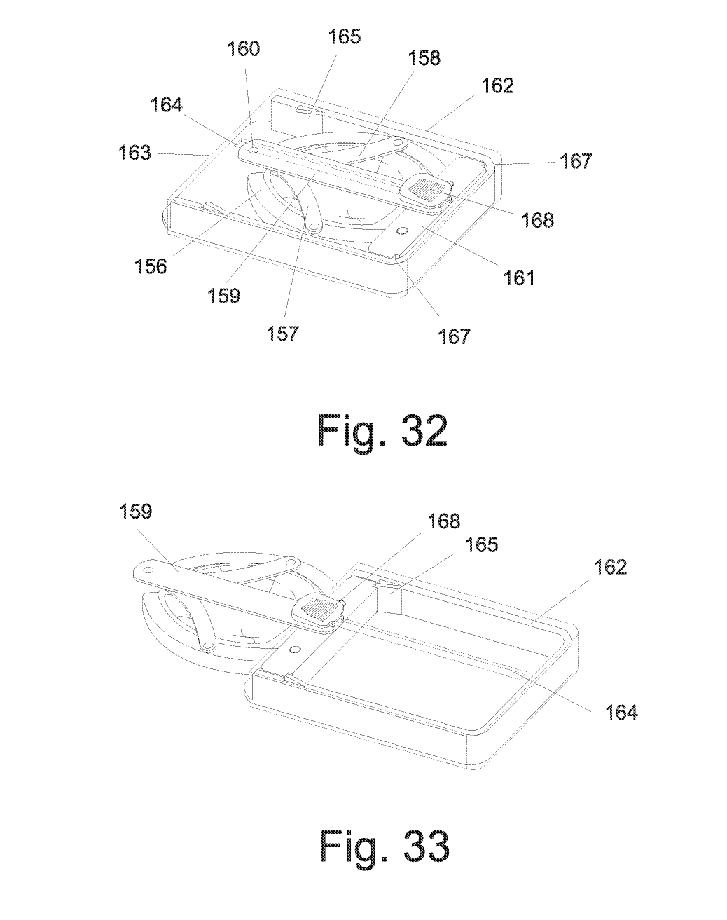

The unfolding of the holder can be combined with unpacking thereof, e.g. in the embodiment represented in FIGS. 32-35.

According to this embodiment, a holder 156 ( [symbol] . 32 is similar to the holder 141 shown in FIGS. 30-31. The difference is that strips 157 and 158 similar to strips 147 and 148 are attached to a rod 159 by one hinge joint 160, as shown in FIG. 32. Furthermore, the rod 159, in contrast to the rod 150 (FIG. 30), passes above a frame 161 similar to the frame 144 (FIG. 30) and rests thereupon.

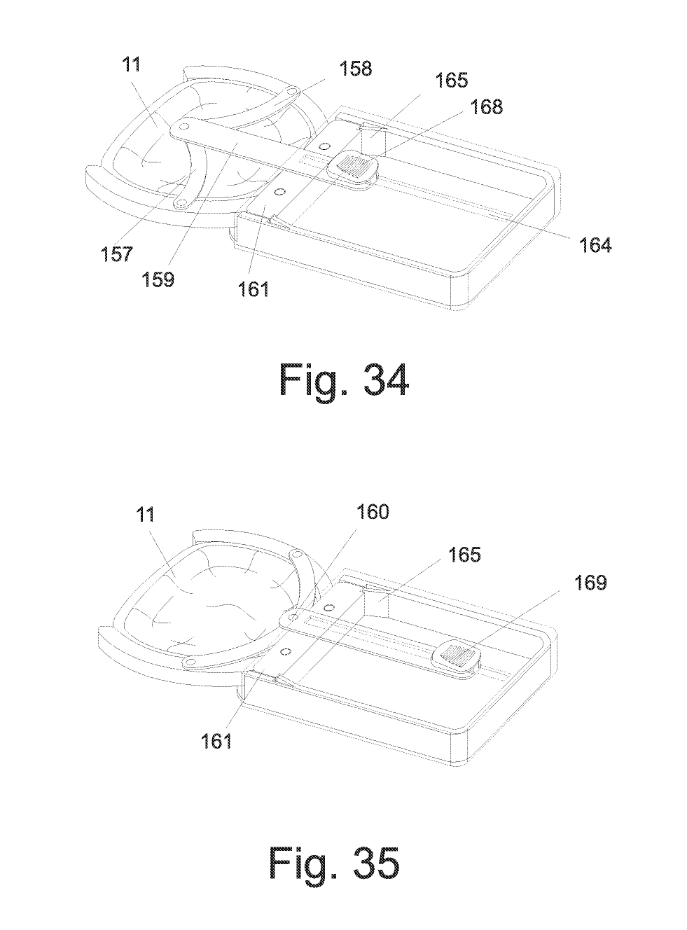

The holder 156 is received in a flat box 162 (shown transparent for descriptive reasons), which includes one open end 163 and a central through slot 164 formed on one side of the box 162, as shown in FIG. 32. Locking devices 165 formed as offbending plates are placed on the inner side of the box 162 in close vicinity of the open end 163 thereof. Said locking devices are adapted for the mechanical engagement with cutouts 167 formed on the frame 161 (FIGS. 33-34), and restrict the displacement of the holder 156 back into the box 162.

The rest 168 is a key or a button with shape and size comfortable to be affected by a digit. The rest 168 is placed outside the box 162 and is connected to the holder 156 by a rod 169 placed in the slot 164.

The device is operated as shown in FIGS. 33-35. The user holds the device in hand in such manner that the open end 163 of the box is faced forward, away from the user, and sets his thumb on the rest 168. Then the user moves said thumb forward, away from the user. When the rest 168 reaches the end of the slot 164, the frame 161 is located near the open end 163, as shown in FIG. 33. Afterwards, the user affects the rest 168, displacing it in a direction towards the user. Due to the fact that the frame 161 is locked on the open end 163 by locking devices 165, the displacement of the rest 168 and, subsequently, the rod 159 leads to rotation of strips 157 and 158 (FIG. 34), which displace the arc-shaped bands in opposite directions and thus expand the area defined by the holder 156. In the position where the frame 161 restricts the further displacement of the hinge joint 160, as shown in FIG. 35, the condom 11 is stretched and ready to be put on. Then the user puts the condom 11 on, holding the box in one hand and displacing it towards the base of the penis. After the condom is put on, the holder can be removed in transverse direction.

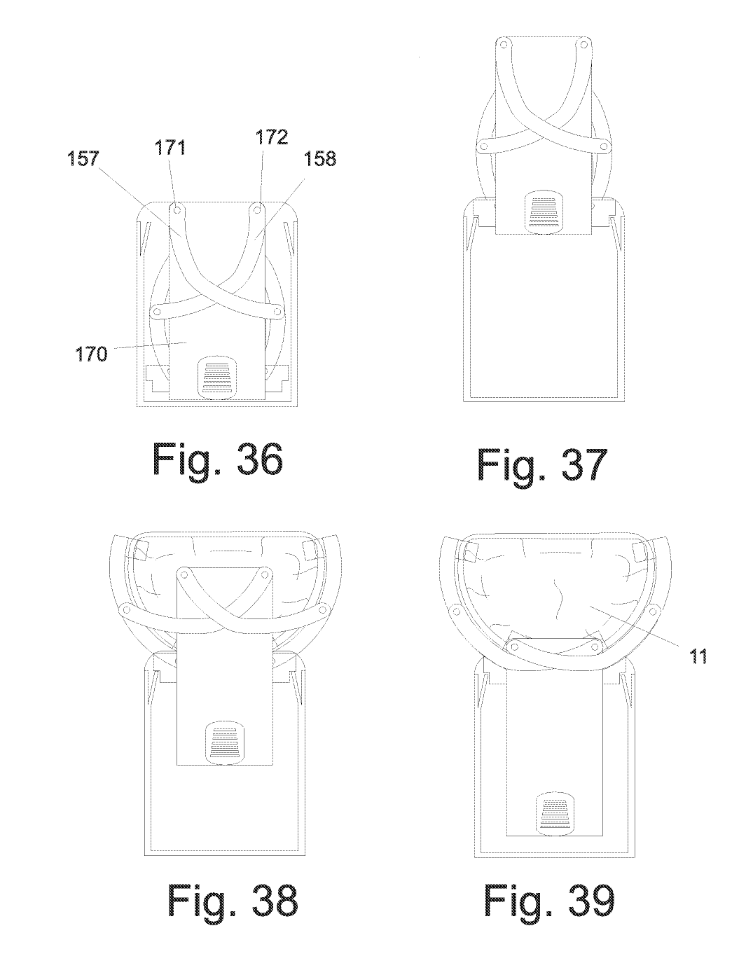

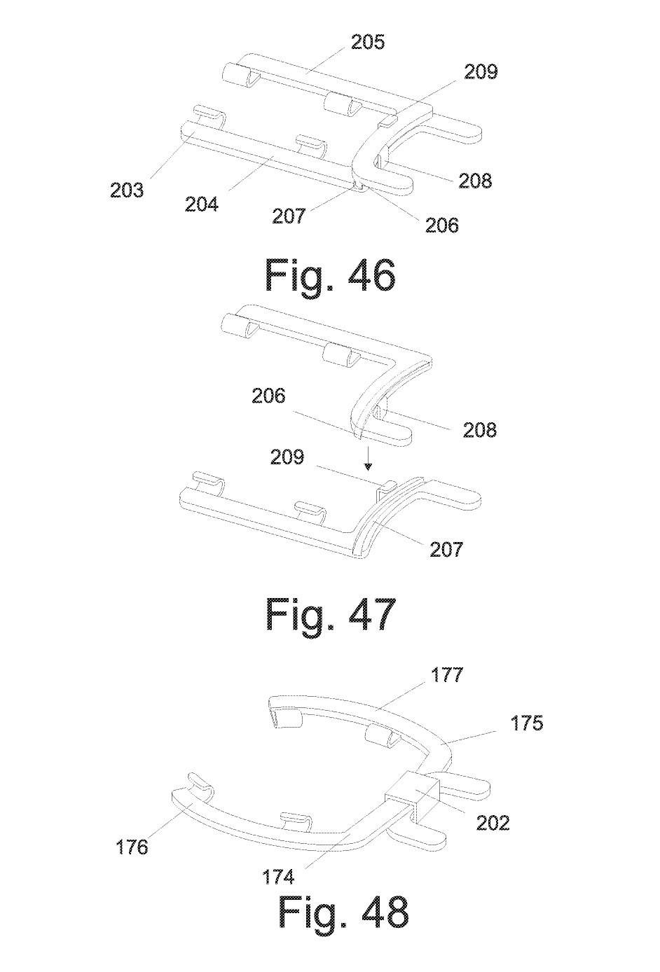

In the design described above, a somewhat different connection of strips 157 and 158 to each other can be used, as shown in FIGS. 36-39. A plate 170 is used instead of the rod 159, and strips 157 and 158 are arranged crosswise and attached to said plate by two hinge joints 171 and 172. The device shown in FIGS. 46-48 is operated similarly to the device shown in FIGS. 33-35.

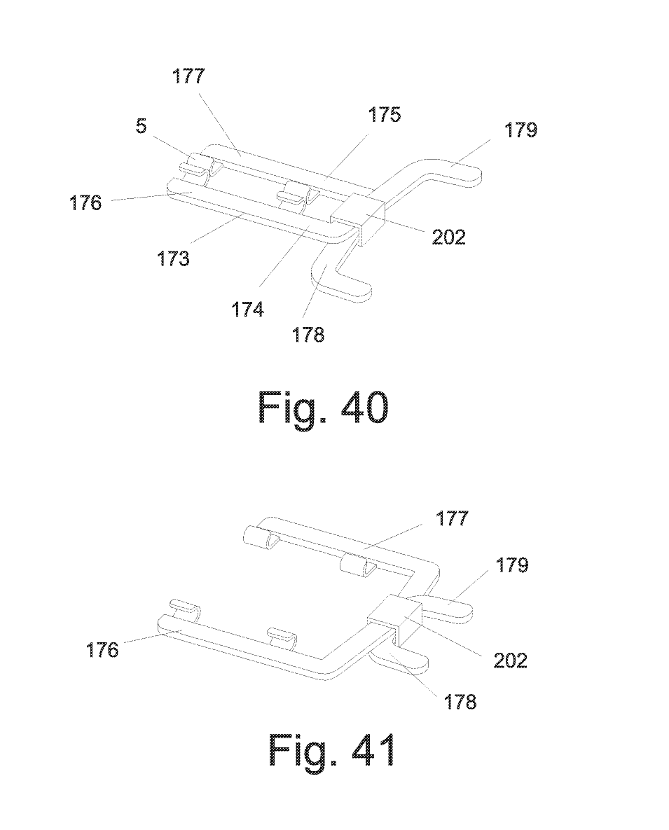

An embodiment is also possible, as shown in FIGS. 40-41, characterized in that the holder 173 is provided with two bands 174 and 175 arranged crosswise and moveably connected to each other by a guideway 202 provided with an opening of rectangular section embracing said bands at the junction thereof.

Stripes 174 and 175 are mostly Z-shaped. Fasteners 5 are formed on one end 176 and 177 of each band, and the other end is adapted to be affected by the digits of a hand.

The device is operated as follows: The user takes the holder 173 in hand, holding it with by the ends 178 and 179 by the digits, then he moves the digits towards each other. Ends 176 and 177 are therefore displaced in opposite directions. In the position of the holder shown in FIG. 41, the condom (not shown) is stretched and ready to be put on.

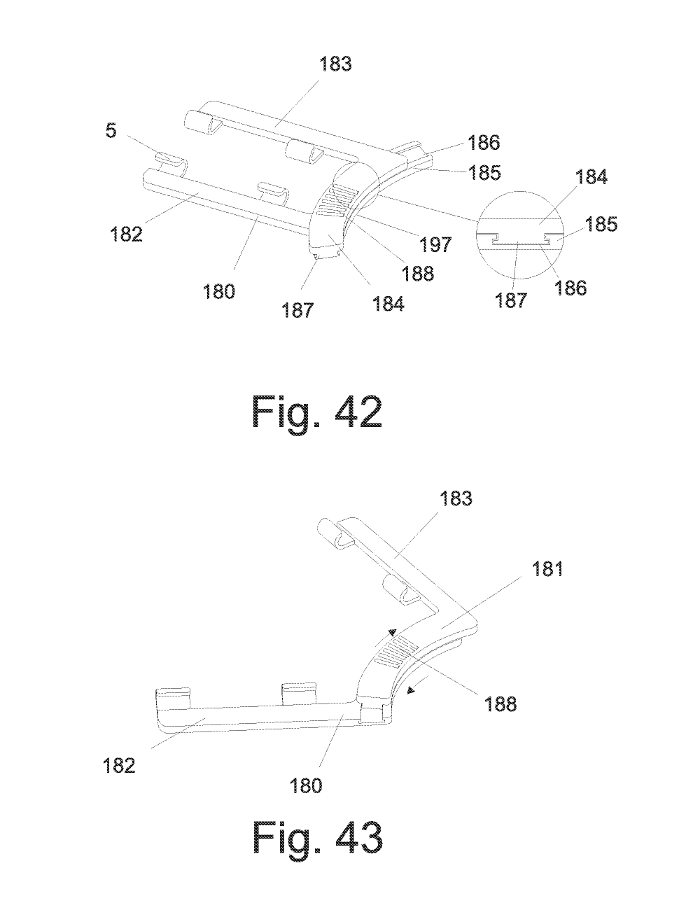

In yet another embodiment represented in FIGS. 42-43, a holder includes two mainly L-shaped bands 180 and 181. Fasteners 5 are placed on sides 182 and 183 of each band 180 and 181, and sides 184 and 185 of said bands are arc-shaped and moveably connected to each other. The side 185 includes a slot 186, and the side 184 includes a dovetail lug 187 adapted to be placed into said slot 186 of the same shape; said slot is used as a guideway for the lug 187 when bands 180 and 181 are displaced with respect to each other.

A ribbed rest section 188 is formed on the side 184; said rest section provides improved mechanical engagement of a digit and the surface of the band. A rest section 197 (not shown in figures) is formed on the side 185 opposite to the rest section 188.

The device is operated as follows: The user takes the holder with two digits (the index finger and the thumb), then displaces the band 181 with respect to the band 180 by pressing the rest sections 188 and 197, as shown with arrows in FIG. 43. Sides 182 and 183 are displaced in opposite directions, thus stretching the condom (not shown). The above holder is the most compact.

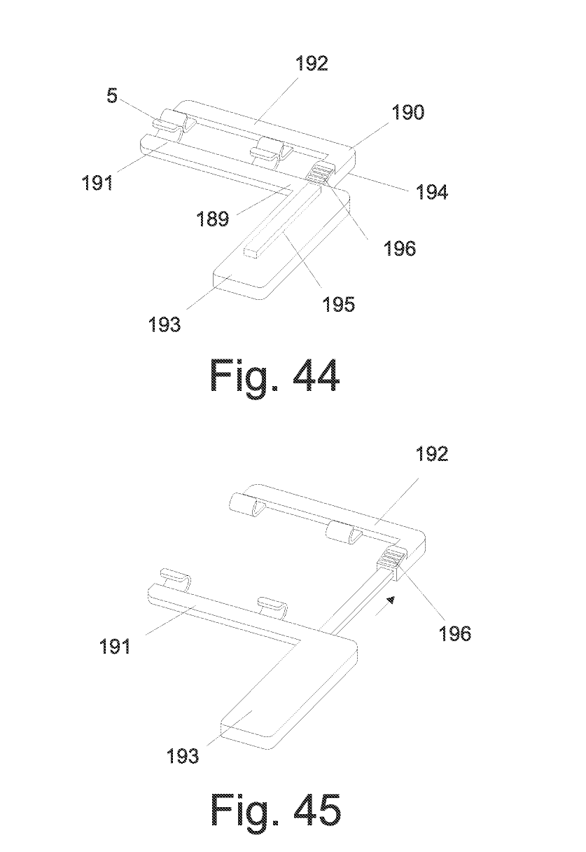

In the embodiment represented in FIGS. 44-45, the holder also includes two L-shaped bands 189 and 190. Fasteners 5 are placed on sides 191 and 192 of said bands, and sides 193 and 194 of said bands are arc-shaped and connected to each other adapted for translational displacement. The side 193 of the band 189 is a plate adapted to be comfortably held in hand; said plate includes an opening 195, shown with a dashed line in FIG. 44; the side 194 of the band 190 is placed therein. The side 194 is provided with a ribbed rest 196.

When putting on the condom, the user holds the holder by the side 193 used as a handle, and affects the rest 196 with a digit (preferrably with the thumb) in a direction shown with an arrow in FIG. 45. Sides 191 and 192 are therefore brought apart, as shown in FIG. 45), and the condom (not shown) is stretched.

In the holder design represented above in FIGS. 40-41, the connection of bands can be different. As shown in FIGS. 46-47, a holder 203 includes two bands 204 and 205 provided with a lug 206 and a slot 207 respectively. The lug 206 is placed in the slot 207 which serves as a guideway for said lug. Furthermore, bands 204 and 205 include L-shaped lugs 208 and 209 placed at the spot where said bands are connected, faced towards each other and embracing bands 204 and 205 as shown in FIG. 46, thus preventing the displacement of bands in any direction other than the condom plane. The device is operated similarly to the device described with reference to FIGS. 40-41.

Furthermore, the holder 173 shown in FIGS. 40-41 can include ends 176 and 177 of a different shape, e.g. as shown in FIG. 48, said ends can be arc-shaped. Bands 174 and 175 are also moveably connected to each other by a guideway 202, and ends 176 and 177 are arranged mainly at an angle to the junction of bands 174 and 175 which in this case includes the guideway 202.

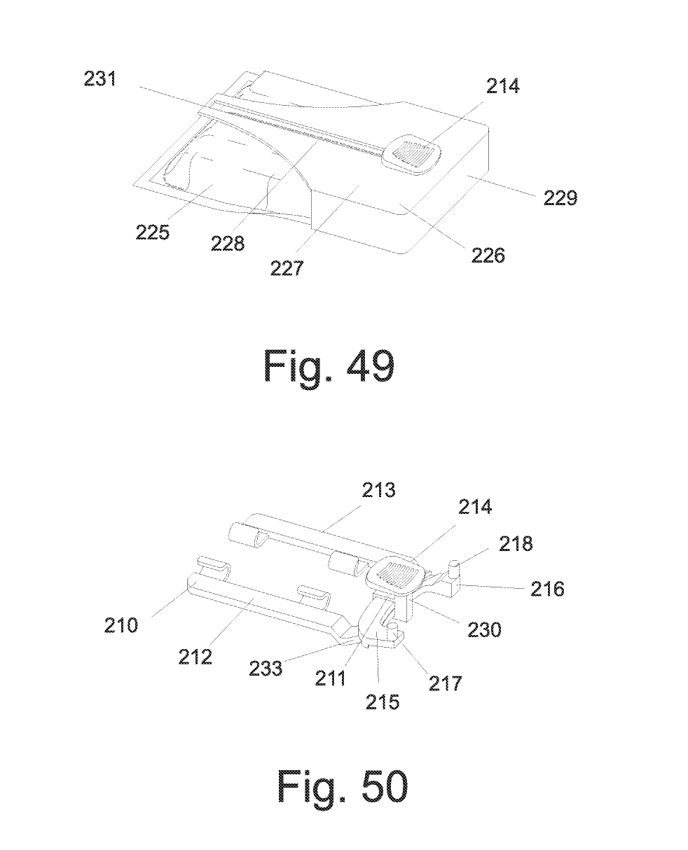

The unfolding of the holder similar to that represented in FIGS. 40-41 can be combined with unpacking thereof, e.g. in the embodiment represented in FIGS. 49-54.

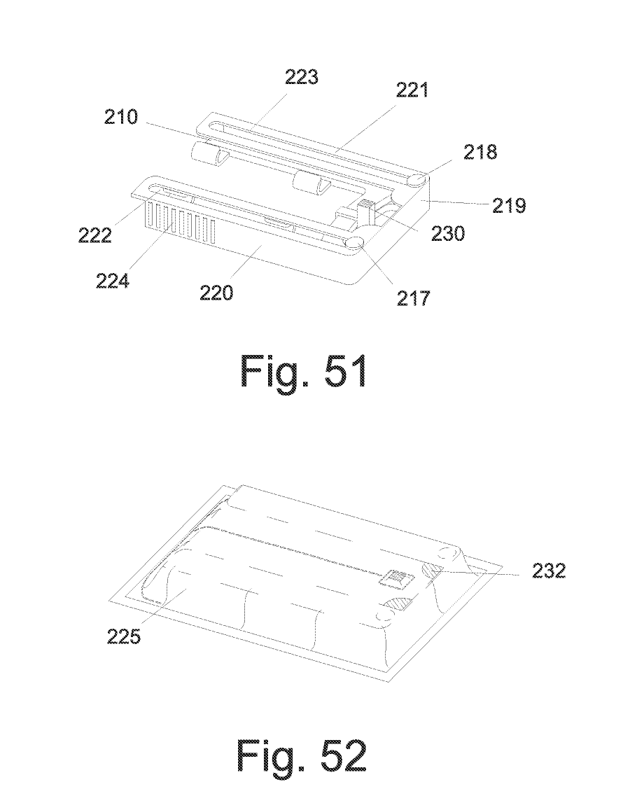

According to this embodiment, a holder 210 ( [symbol] . 50 is similar to the holder 173 shown in FIGS. 40-41. The difference is that a guideway 211 with an opening of rectangular section connecting bands 212 and 213 of the holder 210 is provided with a rest 214 connected to the guideway 211 by a rod 230, and rods 217 and 218 are formed on the ends 215 and 216 of said bands. The holder 210 is placed in a U-shaped frame 219, as shown in FIG. 51, which is faced towards the holder 210 by the inner side thereof, embracing said holder. Sides 220 and 221 of the frame 219 are L-shaped, slots 222 and 223 with rods 217 and 218 moveably placed therein are placed on one side of said profile, and ribbed sections 224 are formed on the other side of said profile.

The holder 210 placed in the frame 219 is received in a package 225 provided with weakened lines facilitating the unpacking; the preferrable arrangement of said weakened lines is shown in FIG. 52. The package 225 is connected (e.g. glued) to a case 226 formed as a plate 227 with a central through slot 228. Connection points of the package 225 with the case are shown in FIG. 52 with hatched sections 232. A flange 229 is formed on one end of the plate 227, and the other end 231 thereof is basically V-shaped. A rest 214 is placed on the side of the plate 227 opposite to that connected to the package, and a rod 230 is moveably placed in the slot 228.

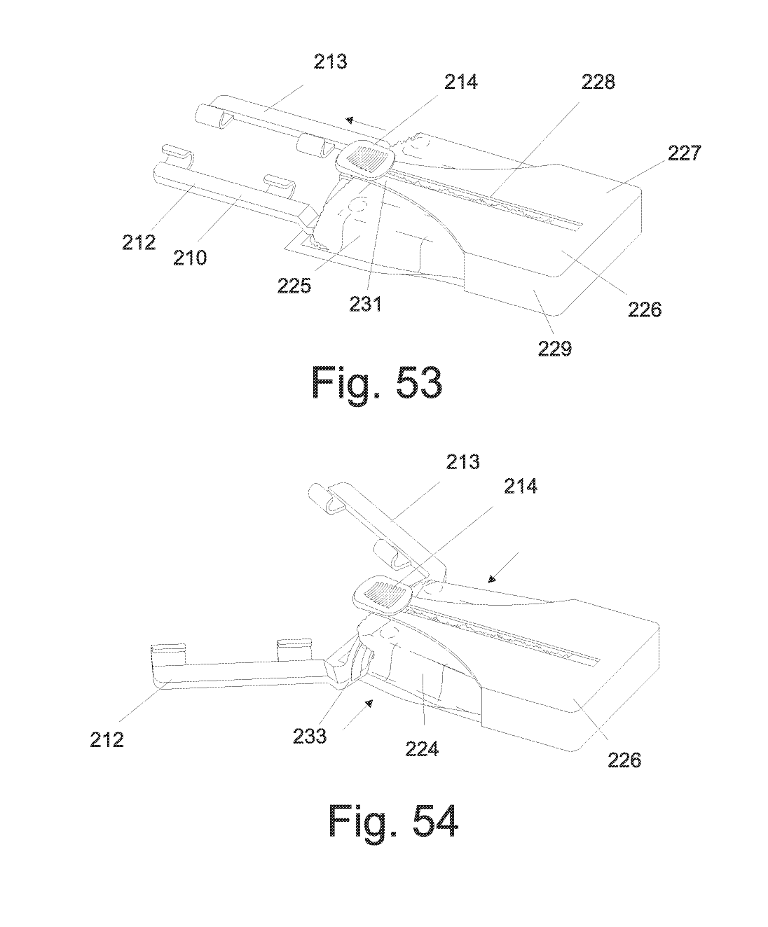

The device is operated as shown in FIGS. 53-54. The user holds the device in hand in such manner that the end 231 of the plate is faced forward, away from the user, and sets his thumb on the rest 214. Then the user moves said thumb forward, away from the user. The holder 210 is therefore displaced, tearing the package 225 along the weakened line. When the rest 214 reaches the end of the slot 228, the holder 210 leaves the package 225 completely, as shown in FIG. 53. Then the user affects the sections 224 through the package 225 using the digits of the same hand holding the holder; said sections are displaced towards each other, as shown with arrows in FIG. 54. Bands 212 and 213 are displaced along the guideway 211 in opposite directions, thus stretching the condom (not shown). Then the user puts the condom on, while holding the holder in one hand and displacing it towards the base of the penis. After the condom is put on, the holder can be removed in transverse direction.

A situation is possible, when the user did not displace the holder completely. In this case, sides 220 and 221 will rest upon the bands 212 and 213 when the sections 224 are pressed, which will hinder the unfolding of the holder. In order to prevent this situation, skewings 233 are formed on the end of bands 212 and 213 of the holder (FIGS. 50, 54); due to this fact, the holder is basically V-shaped on the rear end. In the situation described above, sides 220 and 221 will rest upon the skewings 233, the holder will come out completely and will unfold and stretch the condom according to the method described above without hindrance.

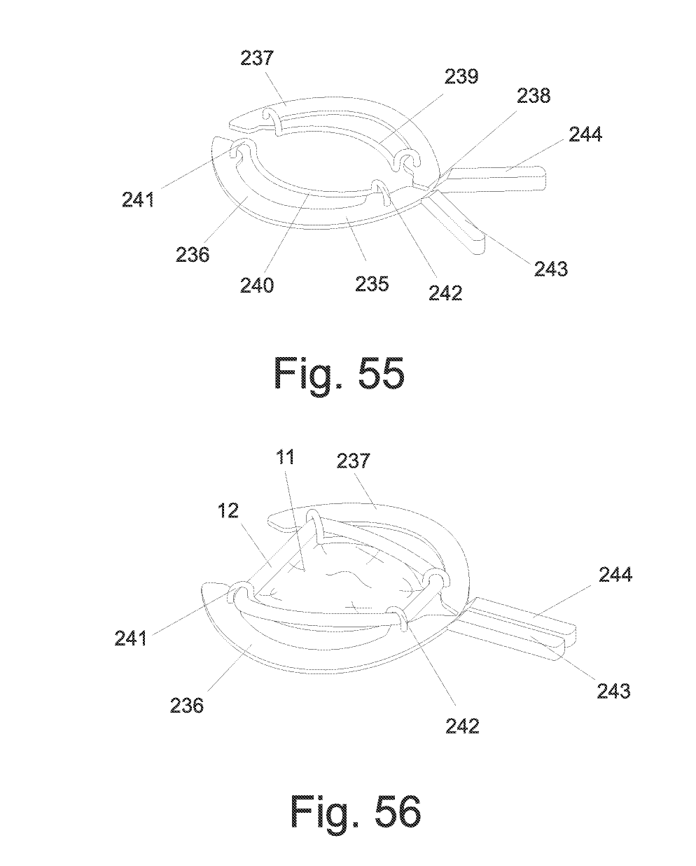

In all embodiments described above, the fasteners can be formed as an arc-shaped wire adapted to embrace the condom ring on the outer side thereof. In the embodiment represented in FIGS. 55-56, a holder 235 includes two arc-shaped bands 236 and 237 connected by a flexible connector 238. A fastener 239 formed as a wire is placed on each band 236, 237. Each fastener 239 includes an arc-shaped part 240 arranged in a plane of bands 236, 237 matching the shape thereof. Ends 241, 242 of each fastener 239 are curved, forming cutouts used to place the rolled condom 11 ring 12 therein. The holder 235 with a condom therein is shown in FIG. 56.

Handles 243 and 244 are attached to bands 236 and 237 on the outer side 13 thereof; said handles can be used for holding the holder when putting the condom on. Said handles 243 and 244 are placed in close proximity to the connector 238 on two sides thereof. The process of putting on a condom with the holder 235 is similar to that described with reference to FIG. 6.

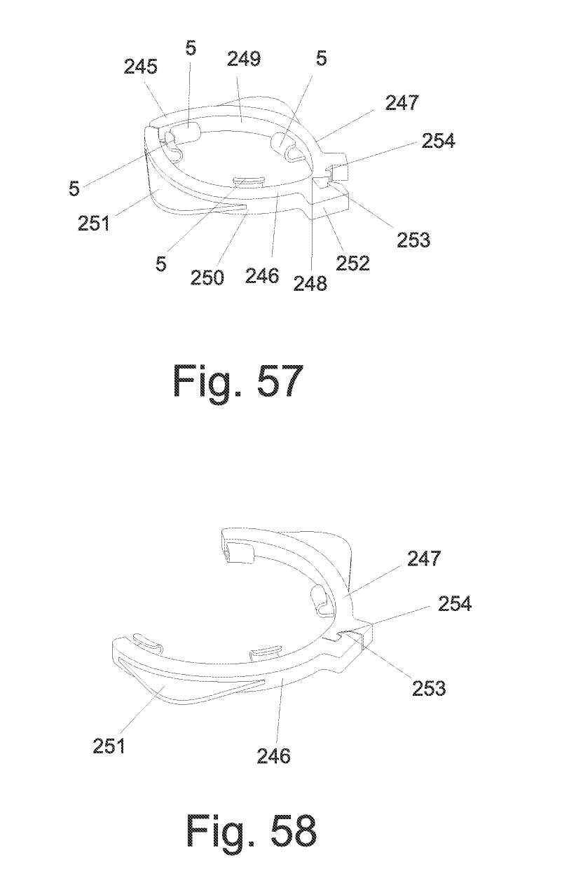

In yet another embodiment represented in FIG. 57-58, a holder 245 includes two arc-shaped bands 246 and 247 connected by displacement means formed as a flexible connector 248. Bands 246 and 247 connect fasteners 5 placed on the inner side 249 of the bands 246 and 247.

Handles 251 formed as plates are attached to bands 246 and 247 on the outer side 250 thereof; said handles can be used for holding the holder when putting the condom on. However, the holder does not necessarily include handles. In such case, the user holds the holder by bands 246 and 247 (not shown).

The band 246 is provided with a L-shaped member 252 placed in close vicinity to the connector 248 and including a lug 253 on the open end thereof, and the band 247 is provided with a slot 254 also placed in close vicinity to the connector 248. By means of said slot 254 and said lug 253, bands 246 and 247 can be fixed with respect to each other, and thus the holder 245 can be locked in unfolded state (FIG. 58).

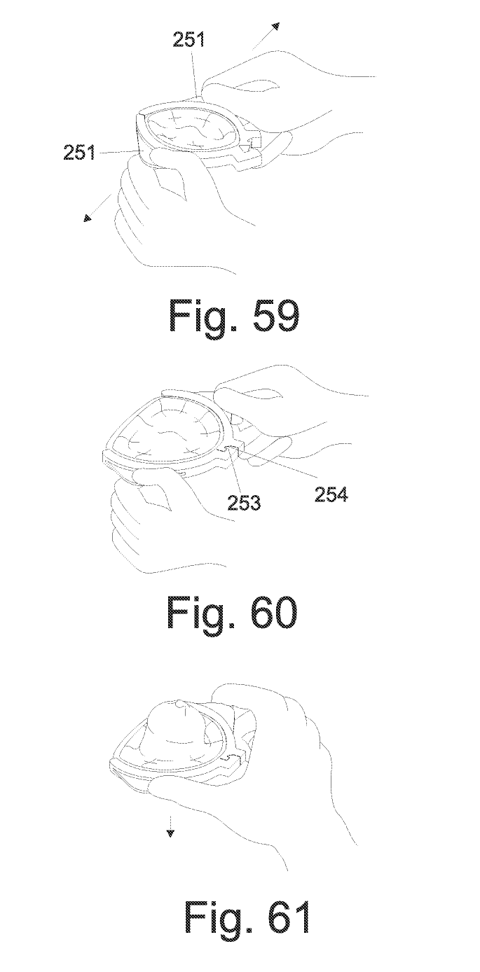

The device is operated as shown in FIG. 59-61. The user takes the device in both hands by handles 251 (FIG. 59) and affects said handles, displacing bands 246 and 247 in opposite directions, rotating said bands with respect to the connector 248. The area defined by the holder 245 is therefore expanded, and the condom 11 is stretched. When bands 246 and 247 are displaced to the limit, the lug 253 is engaged with the slot 254, and the holder 245 is locked in unfolded state (FIG. 60).

Afterwards, the user holds the holder 245 in one hand and displaces it towards the penis root (FIG. 61), then the holder is removed in transverse direction. The holder can be held by two handles and by one handle, due to the fact that bands 246 and 247 are a rigid assembly when locked. Therefore, after the condom is stretched by the holder, one hand is free and the further displacement of the holder can be performed using only one hand. The user can facilitate the process of putting on the condom with the other hand.

Due to the fact that the holder is unfolded by rotating thereof, said holder is more compact in folded state in comparison to holders in which the opposite parts of the connection between fasteners are displaced in parallel opposite directions, e.g. in comparison to the holder shown in FIGS. 40-41. Furthermore, the alteration of shape and placement of handles in this holder (in comparison to the holder shown in FIGS. 7-8) also allows to reduce the holder size. Therefore, the holder described herein provides a comfortable process of putting on the condom by allowing to perform some actions with one hand, and to remove the holder in transverse direction; the size of said holder in assembled state is minimal, which is an advantage when keeping the holder in the user's pocket.

I claim:

1. A condom holder at least partially defining a space in which a flatly arranged condom can be placed, the condom holder comprising fasteners for fixing the condom edge and a connection between said fasteners, the connection including two opposite parts each comprising at least one of said fasteners; and a third part between said two opposite parts, the third part providing for the moveability of said two opposite parts at least away from one another; and a displacement device for displacement of said two opposite parts under the action of mechanical force applied by the user, characterized in that said third part is formed in such manner that said moveability of opposite parts away from one another includes rotation, the condom holder further comprising a holder locking device for locking the holder in the displaced state after said displacement.

2. A holder according to claim 1, characterized in that each of the opposite parts includes at least one band.

3. A holder according to claim 2, characterized in that the band is adapted to bend inside the area defined by the holder.

4. A holder according to claim 3, characterized in that the band is provided with a stopper restricting the bending inside the area defined by the holder.

5. A holder according to claim 3, characterized in that said bending possibility is provided by a flexible connector.

6. A holder according to claim 3, characterized in that said bending possibility is provided by a hinge joint.

7. A holder according to claim 1, characterized in that said rotation possibility is provided by a flexible connector.

8. A holder according to claim 1, characterized in that said rotation possibility is provided by a hinge joint.

9. A holder according to claim 1, characterized in that said third part includes a flexible connector.

10. A holder according to claim 1, characterized in that said third part includes a hinge joint.

11. A holder according to claim 1, characterized in that it includes two levers, wherein the first arms of these levers are formed by said two opposite parts, and their second arms are formed by said displacement device.

12. A holder according to claim 1, characterized in that the third part includes two transfer members, each hinged to one of the two opposite parts; wherein the two transfer members are rotatably connected to each other.

13. A holder according to claim 1, characterized in that the fasteners are noose-shaped.

14. A holder according to claim 13, characterized in that the fasteners are adapted to hold the rolled edge of the condom on the outer side of the condom.

15. A condom holder at least partially defining a space in which a flatly arranged condom can be placed, the condom holder comprising two levers, wherein the first arm of each lever includes at least one fastener for fixing the condom edge, said levers are connected to each other in such a manner that their first arms can be displaced at least away from each other, and two second arms of these levers are adapted to be affected mechanically by the user in order to provide for said displacement, the condom holder further comprising. a holder locking device for locking the holder in the displaced state after said displacement.

16. A holder according to claim 15, characterized in that said levers have a common pivot at which said levers are connected.

17. A holder according to claim 15, characterized in that said levers are connected to each other with a connector, and the pivots thereof are located on the ends of said connector.

18. A holder according to claim 15, characterized in that the levers are adapted to be affected by the digits of one hand in the condom plane.

19. A holder according to claim 15, characterized in that the lever arms adapted to be affected mechanically by the user are rotatably connected to said first lever arms and are adapted to be placed along said first arms.

20. A holder according to claim 19, characterized in that it is received in a box open at one end, the box having including a slot defining a guideway; wherein the holder is provided with a rest connected thereto by means of a part moveably placed in said slot; the rest is placed outside the box, and the lever arms adapted to be affected mechanically by the user are rotatably connected to the box with rotation ability.

21. A holder according to claim 5, characterized in that each lever includes at least one band.

22. A holder according to claim 21, characterized in that the band is adapted to bend inside the area defined by the holder.

23. A holder according to claim 22, characterized in that the band is provided with a stopper restricting the bending inside the area defined by the holder.

24. A holder according to claim 22, characterized in that said bending possibility is provided by means of a flexible connector.

25. A holder according to claim 22, characterized in that said bending possibility is provided by means of a hinge joint.

26. A condom holder at least partially defining a space in which a flatly arranged condom can be placed, that comprises two single-arm levers, each lever's arm having a first section including at least one fastener for fixing the condom edge, wherein said levers are connected to each other in such a manner that their first sections can be displaced at least away from each other, each lever's arm having another section adapted to be affected mechanically by the user in order to provide for said displacement, the condom holder further comprising a holder locking device for locking the holder in the displaced state after said displacement.

27. A holder according to claim 25, characterized in that said levers intersect in such manner that the section of each lever adapted to be affected mechanically by the user is on the side of the other lever.

28. A holder according to claim 25, characterized in that the sections of the levers adapted to be affected mechanically by the user are placed closer to the pivot than the first sections.

29. A holder according to claim 25, characterized in that the first sections of the levers are placed closer to the pivot than sections of the levers adapted to be affected mechanically by the user.

30. A holder according to claim 25, characterized in that said levers have a common pivot at which said levers are connected.

31. A holder according to claim 25, characterized in that each lever includes at least one band.

32. A holder according to claim 31, characterized in that the band is adapted to bend inside the area defined by the holder.

33. A holder according to claim 32, characterized in that the band is provided with a stopper restricting the bending inside the area defined by the holder.

34. A holder according to claim 32, characterized in that said bending possibility is provided by means of a flexible connector.

35. A holder according to claim 32, characterized in that said bending possibility is provided by means of a hinge joint.

36. A condom holder at least partially defining a space in which a flatly arranged condom can be placed, the condom holder having two opposite parts each including at least one fastener for fixing the condom edge; wherein the two opposite parts are connected to each other and adapted to be displaced at least away from each other, characterized in that each of the two opposite parts includes a transfer member hinged to said part, and two transfer members of the two opposite parts of the holder are rotatably connected to each other.

37. A holder according to claim 36, characterized in that it is provided with a locking device for locking said opposite parts in the displaced state after said displacement.

38. A holder according to claim 36, characterized in that said transfer members are hinged to each other.

39. A holder according to claim 36, characterized in that said transfer members are hinged to the end of a rod adapted to displace along the guideway which is also used to connect parts of the holder to each other due to the fact that said parts of the holder are hinged to said guideway by ends of said parts.

40. A holder according to claim 39, characterized in that it is received in a box open at one end and including a slot used as a guideway; the holder is provided with a rest connected to the end of the rod by a part moveably placed in said slot; the rest is placed outside the box.

41. A holder according to claim 36, characterized in that said opposite parts include at least one band.

42. A holder according to claim 41, characterized in that the band is adapted to bend inside the area defined by the holder.

43. A holder according to claim 42, characterized in that the band is provided with a stopper restricting the bending inside the area defined by the holder.

44. A condom holder at least partially defining a space in which a flatly arranged condom can be placed, the condom holder comprising two angular parts moveably connected to each other and arranged in such manner that their first sections, each including at least one fastener for holding the condom edge, are positioned opposite each other, and their second sections, angled with respect to the first sections, extend along each other, wherein said connection of the angular parts is arranged at their second sections in such a manner as to provide for, said displacement of the angular parts by displacing second sections thereof along each other with corresponding displacement of first sections thereof at least away from each other.

45. A holder according to claim 47, characterized in that it is provided with a holder locking device for locking the angular parts in the displaced state after said displacement.

46. A holder according to claim 44, characterized in that said angular parts include corresponding handles on the ends of second sections thereof; said handles being used to facilitate affecting said angular parts mechanically for their displacement.

47. A holder according to claim 46, characterized in that said handles are connected to the angular parts in such a manner as to allow their rotation and positioning along the angular parts.

48. A holder according to claim 47, characterized in that said handles are adapted to be positioned along the first sections.

49. A holder according to claim 48, characterized in that said angular parts intersect in such manner that the handle of each angular part is positioned on the side of the other angular part.

50. A holder according to claim 48, characterized in that said second sections are located in one plane.

51. A holder according to claim 48, characterized in that said second sections are located one above the other.

52. A holder according to claim 44, characterized in that it includes a guideway comprising an opening embracing the second sections.

53. A holder according to claim 44, characterized in that each angular part includes at least one band.

54. A holder according to claim 53, characterized in that the band is adapted to bend inside the area defined by the holder.

55. A holder according to claim 54, characterized in that the band is provided with a stopper restricting the bending inside the area defined by the holder.

56. A holder according to claim 54, characterized in that said bending possibility is provided by means of a flexible connector.

57. A holder according to claim 54, characterized in that said bending possibility is provided by means of a hinge joint.

58. A method of putting on a flatly arranged condom held by a holder at least partially defining the area in which the condom is placed, the condom holder including two opposite parts each including at least one fastener for holding the condom edge and a displacement device for displacement of said two opposite parts under the action of mechanical force applied by the user, wherein the user holds the holder in his hand, displaces said two opposite parts until the condom is stretched, and then puts on the condom by moving the holder towards the base of the penis, characterized in that the user effects said displacement by affecting said two opposite parts with at least two digits of his hand holding the holder.

59. A method according to claim 58, characterized in that the two opposite parts are locked in the displaced state after said displacement.

60. A method according to claim 59, characterized in that the user locks the two opposite parts with a digit of his hand holding the holder.

61. A method according to claim 58, characterized in that said affection is pressing said opposite parts in the condom plane.

62. A method according to claim 58, characterized in that the pressing is performed by at least the thumb and the index finger of the hand holding the holder.

63. A method according to claim 58, characterized in that the displacement is directed away from the palm of the user's hand.

64. A method according to claim 58, characterized in that the displacement is directed towards the palm of the user's hand.

65. A method according to claim 58, characterized in that the user affects levers formed by said opposite parts.

66. A method according to claim 68, characterized in that the user displaces first arms of said levers away from each other and affects the second arms of the levers with two digits of the same hand by displacing said arms towards each other.

67. A method according to claim 65, characterized in that the levers are single-arm.

68. A method according to claim 67, characterized in that the user displaces first sections of the arms of said levers away from each other by affecting the second sections of the arms of the levers, placed closer to the pivot than their first sections, by displacing the second sections of said arms towards each other.

69. A method according to claim 58, characterized in that user displaces the first sections of the angular parts of the holder formed by said opposite parts away from each other by affecting the second sections of the angular parts with two digits of the same hand by displacing said sections along each other.

70. A method according to claim 69, characterized in that said affection of the second sections of the angular parts is performed by affecting handles on the ends thereof.

71. A method according to claim 58, characterized in that said displacement of the opposite parts is performed by affecting the connection point of transfer members by a digit of the same hand.

72. A method according to claim 71, characterized in that user holds the base connected to the holder and adapted to be held in hand, and displaces the holder by affecting the connection point of the transfer members with a digit of his hand holding the base.

73. A condom holder at least partially defining a space in which a flatly arranged condom can be placed, the condom holder comprising fasteners used for fixing the condom edge, a connection between said fasteners, the connection including two opposite parts connected to each other by a displacement device providing moveability thereof at least away from each other, each of said two opposite parts including at least one of said fasteners, and a locking device locking the holder in the displaced state after said displacement, characterized in that said displacement device is formed in such manner that said moveability of opposite parts in opposite directions includes rotation.

74. A holder according to claim 73, characterized in that each opposite part includes a band having its one end connected to the displacement means, wherein its other end is free.

75. A holder according to claim 73, characterized in that the displacement device includes a hinge joint.

76. A method of putting on the flatly arranged condom held by a holder at least partially defining the area in which the condom is placed, the condom holder including two opposite parts each including at least one fastener for holding the condom edge, a displacement device between said two parts for providing moveability thereof under the action of mechanical force applied by the user, and a locking device keeping the holder in the displaced state after said displacement, wherein the user holds the holder in both hands and displaces said opposite parts until the locking device locks the opposite parts in the displaced state, then the user takes the holder in one hand, puts the condom on by displacing the holder towards the penis root, and removes the holder, characterized in that said opposite parts are rotated with respect to each other in the condom plane, and the holder is removed in the transverse direction.
